SEQUENCE LISTING (14 sequences)

SEQ ID NO: 1 - DNA, 1765 bp, Sulfolobus solfataricus
  Beta-glycosidase of Sulfolobus solfataricus
ggatcaatactaggaggagtagcatataattacgttacacaattttataacccaatatattcaatagaccttatgcttatcctatcctctattctaagattctcggtatctcccctattcttgaccataaaagatactcgctcaaagcttaaataatattaatcataaataaagtcatgtactcatttccaaatagctttaggtttggttggtcccaggccggatttcaatcagaaatgggaacaccagggtcagaagatccaaatactgactggtataaatgggttcatgatccagaaaacatggcagcgggattagtaagtggagatctaccagaaaatgggccaggctactggggaaactataagacatttcacgataatgcacaaaaaatgggattaaaaatagctagactaaatgtggaatggtctaggatatttcctaatccattaccaaggccacaaaactttgatgaatcaaaacaagatgtgacagaggttgagataaacgaaaacgagttaaagagacttgacgagtacgctaataaagacgcattaaaccattacagggaaatattcaaggatcttaaaagtagaggactttactttatactaaacatgtatcattggccattacctctatggttacacgacccaataagagtaagaagaggagattttactggaccaagtggttggctaagtactagaacagtttacgaattcgctagattctcagcttatatagcttggaaattcgatgatctagtggatgagtactcaacaatgaatgaacctaacgttgttggaggtttaggatacgttggtgttaagtccggttttcccccaggatacctaagctttgaactttcccgtagggcaatgtataacatcattcaagctcacgcaagagcgtatgatgggataaagagtgtttctaaaaaaccagttggaattatttacgctaatagctcattccagccgttaacggataaagatatggaagcggtagagatggctgaaaatgataatagatggtggttctttgatgctataataagaggtgagatcaccagaggaaacgagaagattgtaagagatgacctaaagggtagattggattggattggagttaattattacactaggactgttgtgaagaggactgaaaagggatacgttagcttaggaggttacggtcacggatgtgagaggaattctgtaagtttagcgggattaccaaccagcgacttcggctgggagttcttcccagaaggtttatatgacgttttgacgaaatactggaatagatatcatctctatatgtacgttactgaaaatggtattgcggatgatgccgattatcaaaggccctattatttagtatctcacgtttatcaagttcatagagcaataaatagtggtgcagatgttagagggtatttacattggtctctagctgataattacgaatgggcttcaggattctctatgaggtttggtctgttaaaggtcgattacaacactaagagactatactggagaccctcagcactagtatatagggaaatcgccacaaatggcgcaataactgatgaaatagagcacttaaatagcgtacctccagtaaagccattaaggcactaaactttctcaagtctcactataccaaatgagttttcttttaatcttattctaatctcattttcattagattgcaatactttcataccttctatattatttattttgtaccttttgggatc

SEQ ID NO: 2 - protein, 489 aa, Sulfolobus solfataricus
  Beta-glycosidase of Sulfolobus solfataricus
MYSFPNSFRFGWSQAGFQSEMGTPGSEDPNTDWYKWVHDPENMAAGLVSGDLPENGPGYWGNYKTFHDNAQKMGLKIARLNVEWSRIFPNPLPRPQNFDESKQDVTEVEINENELKRLDEYANKDALNHYREIFKDLKSRGLYFILNMYHWPLPLWLHDPIRVRRGDFTGPSGWLSTRTVYEFARFSAYIAWKFDDLVDEYSTMNEPNVVGGLGYVGVKSGFPPGYLSFELSRRAMYNIIQAHARAYDGIKSVSKKPVGIIYANSSFQPLTDKDMEAVEMAENDNRWWFFDAIIRGEITRGNEKIVRDDLKGRLDWIGVNYYTRTVVKRTEKGYVSLGGYGHGCERNSVSLAGLPTSDFGWEFFPEGLYDVLTKYWNRYHLYMYVTENGIADDADYQRPYYLVSHVYQVHRAINSGADVRGYLHWSLADNYEWASGFSMRFGLLKVDYNTKRLYWRPSALVYREIATNGAITDEIEHLNSVPPVKPLRH

SEQ ID NO: 3 - DNA, 1455 bp, Thermotoga petrophila
  alpha-L-arabinofuranosidase of Thermotoga petrophila
atgtcctacaggatagtggttgatccaaaaaaagttgtcaagccgattagtagacacatctacggtcatttcacggaacatctgggaaggtgtatctacggcggaatttatgaagaaggttctccgctctccgatgaaaggggtttcagaaaggacgttctggaggctgtaaagaggataaaagttccgaacttgagatggcccggtggaaactttgtgtcgaactaccactgggaagacggaataggtcccaaagatcagaggcctgtcaggttcgatctcgcctggcaacaggaagagacgaatagatttggaacggacgaattcattgagtactgtcgtgagataggagcagaaccttacatcagtataaacatgggaactggaacactcgacgaagctctccactggcttgaatactgcaatggaaagggtaatacctactacgctcaactcagaagaaagtacggtcatccagaaccttacaacgtaaagttctggggaataggcaacgagatgtacggggaatggcaggtaggccacatgacggcggacgaatacgcaagagccgccaaagaatacacgaaatggatgaaggttttcgatcctacaattaaagcgatcgccgtgggctgtgacgaccctatatggaatctcagggttcttcaagaagcaggtgatgtgattgacttcatatcctaccatttctacacagggtccgaggattactacgaaacagtttccacggtttaccttctcaaagaaagactcatcggagtgaaaaagctcattgatatggtggatactgctagaaagagaggtgtcaaaatcgcccttgatgaatggaacgtatggtacagagtgtccgataacaagctcgaagaaccttacgatctcaaagatggtatctttgcatgtggagtgcttgtacttcttcaaaagatgagcgacatagtcccacttgccaatctcgcacagcttgtaaacgcccttggagctatacacaccgagaaagacggtctcattctcacacccgtttacaaggcttttgaactcatcgtgaatcattccggagaaaagcttgtcaagacccatgttgaatcggagacttacaacatagaaggagtcatgttcatcaacaaaatgcctttctctgtcgagaacgcaccgttccttgatgccgccgcttccatctcagaagatggcaagaaacttttcatcgctgttgtaaactacaggaaagaagacgctttgaaggttccaatcagagtggaaggtctgggacagaaaaaagccaccgtttatacactcacaggtccggacgtgaacgcgagaaacaccatggaaaatccgaacgtcgttgatattacctccgaaaccatcaccgttgacaccgaatttgaacacacgtttaaaccattctcttgcagtgtgattgaggtagaattggagtaa

SEQ ID NO: 4 - protein, 484 aa, Thermotoga petrophila
  alpha-L-arabinofuranosidase of Thermotoga petrophila
MSYRIVVDPKKVVKPISRHIYGHFTEHLGRCIYGGIYEEGSPLSDERGFRKDVLEAVKRIKVPNLRWPGGNFVSNYHWEDGIGPKDQRPVRFDLAWQQEETNRFGTDEFIEYCREIGAEPYISINMGTGTLDEALHWLEYCNGKGNTYYAQLRRKYGHPEPYNVKFWGIGNEMYGEWQVGHMTADEYARAAKEYTKWMKVFDPTIKAIAVGCDDPIWNLRVLQEAGDVIDFISYHFYTGSEDYYETVSTVYLLKERLIGVKKLIDMVDTARKRGVKIALDEWNVWYRVSDNKLEEPYDLKDGIFACGVLVLLQKMSDIVPLANLAQLVNALGAIHTEKDGLILTPVYKAFELIVNHSGEKLVKTHVESETYNIEGVMFINKMPFSVENAPFLDAAASISEDGKKLFIAVVNYRKEDALKVPIRVEGLGQKKATVYTLTGPDVNARNTMENPNVVDITSETITVDTEFEHTFKPFSCSVIEVELE

SEQ ID NO: 5 - DNA, 24 bp, Artificial Sequence
  forward primer to beta-glycosidase of Sulfolobus solfataricus
catatgtactcatttccaaatagc

SEQ ID NO: 6 - DNA, 27 bp, Artificial Sequence
  reverse primer to beta-glycosidase of Sulfolobus solfataricus
ctcgagttagtgccttaatggctttac

SEQ ID NO: 7 - DNA, 31 bp, Artificial Sequence
  forward primer to alpha-L-arabinofuranosidase of Thermotoga petrophila
catatgatgtcctacaggatagtggttgatc

SEQ ID NO: 8 - DNA, 27 bp, Artificial Sequence
  reverse primer to alpha-L-arabinofuranosidase of Thermotoga petrophila
ctcgagctccaattctacctcaatcac

SEQ ID NO: 9 - DNA, 5310 bp, Artificial Sequence
  pET-24a(+) vector
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 10 - DNA, 5443 bp, Artificial Sequence
  pET-21a vector
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggctagcatgactggtggacagcaaatgggtcgcggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 7002 bp, Artificial Sequence
  pET-24a/beta-glycosidase vector
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagggatcaatactaggaggagtagcatataattacgttacacaattttataacccaatatattcaatagaccttatgcttatcctatcctctattctaagattctcggtatctcccctattcttgaccataaaagatactcgctcaaagcttaaataatattaatcataaataaagtcatgtactcatttccaaatagctttaggtttggttggtcccaggccggatttcaatcagaaatgggaacaccagggtcagaagatccaaatactgactggtataaatgggttcatgatccagaaaacatggcagcgggattagtaagtggagatctaccagaaaatgggccaggctactggggaaactataagacatttcacgataatgcacaaaaaatgggattaaaaatagctagactaaatgtggaatggtctaggatatttcctaatccattaccaaggccacaaaactttgatgaatcaaaacaagatgtgacagaggttgagataaacgaaaacgagttaaagagacttgacgagtacgctaataaagacgcattaaaccattacagggaaatattcaaggatcttaaaagtagaggactttactttatactaaacatgtatcattggccattacctctatggttacacgacccaataagagtaagaagaggagattttactggaccaagtggttggctaagtactagaacagtttacgaattcgctagattctcagcttatatagcttggaaattcgatgatctagtggatgagtactcaacaatgaatgaacctaacgttgttggaggtttaggatacgttggtgttaagtccggttttcccccaggatacctaagctttgaactttcccgtagggcaatgtataacatcattcaagctcacgcaagagcgtatgatgggataaagagtgtttctaaaaaaccagttggaattatttacgctaatagctcattccagccgttaacggataaagatatggaagcggtagagatggctgaaaatgataatagatggtggttctttgatgctataataagaggtgagatcaccagaggaaacgagaagattgtaagagatgacctaaagggtagattggattggattggagttaattattacactaggactgttgtgaagaggactgaaaagggatacgttagcttaggaggttacggtcacggatgtgagaggaattctgtaagtttagcgggattaccaaccagcgacttcggctgggagttcttcccagaaggtttatatgacgttttgacgaaatactggaatagatatcatctctatatgtacgttactgaaaatggtattgcggatgatgccgattatcaaaggccctattatttagtatctcacgtttatcaagttcatagagcaataaatagtggtgcagatgttagagggtatttacattggtctctagctgataattacgaatgggcttcaggattctctatgaggtttggtctgttaaaggtcgattacaacactaagagactatactggagaccctcagcactagtatatagggaaatcgccacaaatggcgcaataactgatgaaatagagcacttaaatagcgtacctccagtaaagccattaaggcactaaactttctcaagtctcactataccaaatgagttttcttttaatcttattctaatctcattttcattagattgcaatactttcataccttctatattatttattttgtaccttttgggatccatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 12 - DNA, 6821 bp, Artificial Sequence
  pET-21a/alpha-L-arabinofuranosidase vector
ggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgtcctacaggatagtggttgatccaaaaaaagttgtcaagccgattagtagacacatctacggtcatttcacggaacatctgggaaggtgtatctacggcggaatttatgaagaaggttctccgctctccgatgaaaggggtttcagaaaggacgttctggaggctgtaaagaggataaaagttccgaacttgagatggcccggtggaaactttgtgtcgaactaccactgggaagacggaataggtcccaaagatcagaggcctgtcaggttcgatctcgcctggcaacaggaagagacgaatagatttggaacggacgaattcattgagtactgtcgtgagataggagcagaaccttacatcagtataaacatgggaactggaacactcgacgaagctctccactggcttgaatactgcaatggaaagggtaatacctactacgctcaactcagaagaaagtacggtcatccagaaccttacaacgtaaagttctggggaataggcaacgagatgtacggggaatggcaggtaggccacatgacggcggacgaatacgcaagagccgccaaagaatacacgaaatggatgaaggttttcgatcctacaattaaagcgatcgccgtgggctgtgacgaccctatatggaatctcagggttcttcaagaagcaggtgatgtgattgacttcatatcctaccatttctacacagggtccgaggattactacgaaacagtttccacggtttaccttctcaaagaaagactcatcggagtgaaaaagctcattgatatggtggatactgctagaaagagaggtgtcaaaatcgcccttgatgaatggaacgtatggtacagagtgtccgataacaagctcgaagaaccttacgatctcaaagatggtatctttgcatgtggagtgcttgtacttcttcaaaagatgagcgacatagtcccacttgccaatctcgcacagcttgtaaacgcccttggagctatacacaccgagaaagacggtctcattctcacacccgtttacaaggcttttgaactcatcgtgaatcattccggagaaaagcttgtcaagacccatgttgaatcggagacttacaacatagaaggagtcatgttcatcaacaaaatgcctttctctgtcgagaacgcaccgttccttgatgccgccgcttccatctcagaagatggcaagaaacttttcatcgctgttgtaaactacaggaaagaagacgctttgaaggttccaatcagagtggaaggtctgggacagaaaaaagccaccgtttatacactcacaggtccggacgtgaacgcgagaaacaccatggaaaatccgaacgtcgttgatattacctccgaaaccatcaccgttgacaccgaatttgaacacacgtttaaaccattctcttgcagtgtgattgaggtagaattggagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 13 - DNA, 1647 bp, Artificial Sequence
  GroEL sequence
atggcagctaaagacgtaaaattcggtaacgacgctcgtgtgaaaatgctgcgcggcgtaaacgtactggcagatgcagtgaaagttaccctcggtccgaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctccaaagcgaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccgtgctctgattctaaagcgattgctcaggttggtaccatctccgctaactccgacgaaaccgtaggtaaactgatcgcagaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgcaaaagcaggtaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtaggtgctgctaccgaagttgaaatgaaagagaaaaaagcacgcgttgaagatgccctgcacgcgacccgtgcagcggtagaagagggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgagtgcatggttaccgacctgccgaaaaacgatgcagctgacttaggcgctgctggcggtatgggcggcatgggtggcatgggcggcatgatgtaa

SEQ ID NO: 14 - DNA, 294 bp, Artificial Sequence
  GroES sequence
atgaatattcgtccattgcatgatcgcgtgatcgtcaagcgtaaagaagttgaaactaaatctgctggcggcatcgttctgaccggctctgcagcggctaaatccacccgtggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaa

(19) United States
(12) Patent Application Publication
(10) Pub. No.: US 2019/0233868 A1
(43) Pub. Date: Aug. 1, 2019

(54) COMPOSITION FOR PRODUCTION OF GINSENOSIDE COMPOUND K COMPRISING HIGH TEMPERATURE alpha-L-ARABINOFURANOSIDASE, AND METHOD FOR PREPARING GINSENDOSIDE COMPOUND K

(71) Applicants: Amorepacific Corporation, Seoul (KR); Konkuk University Industrial Cooperation Corp, Seoul (KR)

(72) Inventors: Junseong PARK, Yongin-si, Gyeonggi-do (KR); Kyeonghwan HWANG, Yongin-si, Gyeonggi-do (KR); Deok-Kun OH, Gwacheon-si, Gyeonggi-do (KR); Kyung-Chul SHIN, Seoul (KR)

(21) Appl. No.: 16/337,688

(22) PCT Filed: Sep. 28, 2017
(86) PCT No.: PCT/KR2017/010849

§ 371 Date: Mar. 28, 2019

(30) Foreign Application Priority Data
Sep. 28, 2016 (KR) 10-2016-0124986

(51) Int. Cl. C12P 33/16; C12N 9/42

(52) U.S. Cl. CPC C12P 33/16; C12Y 302/01021; C12Y 302/01055; C12N 9/2445

(57) ABSTRACT

Disclosed are a composition for production of ginsenoside compound K using a high temperature-β-glycosidase and a high temperature-α-L-arabinofuranosidase, and a method for preparing ginsenoside compound k. The composition for producing ginsenoside compound k and the method for preparing ginsenoside compound k according to one aspect of the present invention allow high temperature-βglycosidase and high temperature-a-L-arabinofuranosidase to exhibit stable activity even at high temperatures, thereby increasing a reaction rate. The composition for producing ginsenoside compound k and the method for preparing ginsenoside compound k according to one aspect of the present invention allow a large quantity of ginsenoside compound k to be produced in a short time, thereby exhibiting an effect of producing a high yield, and thus can be utilized industrially.

### TECHNICAL FIELD

Disclosed are a composition for production of ginsenoside compound K using a high temperature-β-glycosidase and a high temperature-α-L-arabinofuranosidase, and a method for preparing ginsenoside compound K.

### BACKGROUND ART

Ginsenoside compound K (20(S)-protopanaxadiol-20-O-β-D-glucopyranoside; see the following Formula 1) is an intestinal bacterial metabolite of ginseng saponin components. It is produced by hydrolysis of glucose, arabinopyranose and arabinofuranose moieties in ginsenoside Rb1, ginsenoside Rb2, ginsenoside Rc and ginsenoside Rd, which are protopanaxadiol-type saponins.

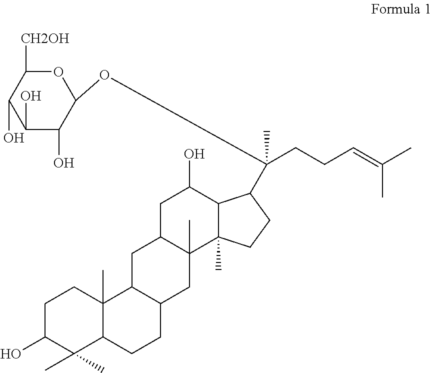

Until now, ginsenoside compound K has been known to have many excellent effects such as immunity enhancement, inhibition of tumor angiogenesis, inhibition of cancer cell infiltration and inhibition of cancer cell proliferation. Accordingly, there is an increasing demand for mass supply of the compound in the field of health foods and cosmetics. Therefore, there is a growing need for producing it stably and efficiently.

The prior art for production of ginsenoside compound K includes methods for preparing compound K by treating diol-type saponins with enzymes such as β-glycosidase (Korean Patent Laid-Open No. 2003-94757), cellulase isolated from a microorganism of the genus Penicillium or β-galactosidase isolated from the genus Aspergillus (Korean Patent No. 377546), naringinase isolated from the genus Penicillium, or pectinase isolated from the genus Aspergillus (Korean Patent No. 418604), etc.

As described above, ginsenoside compound K is mostly produced using mesophilic enzymes active at a temperature in the range of 10 to 50° C. However, since these enzymes act at a low reaction temperature, they are likely to be contaminated with microorganisms and have a low production yield.

In some cases, ginsenoside compound K is produced using high temperature enzymes. However, α-L-arabinofuranosidase shows poor expression and activity, and thus has difficulty in converting ginsenoside Rc to compound K.

Therefore, in order to solve these problems, there is an urgent need to develop enzymes industrially useful for production of ginsenoside compound K and a preparation method using the same.

### SUMMARY OF INVENTION

### Technical Problem

Thus, the present inventors have continuously studied to develop a new method for preparing ginsenoside compound K. An object of the present invention is to provide a composition for production of ginsenoside compound K comprising a high temperature-β-glycosidase derived from a high temperature microorganism, Sulfolobus solfataricus, and an α-L-arabinofuranosidase derived from Thermotoga petrophila and a method for producing ginsenoside compound K using the same.

In one aspect of the present invention, these enzymes are cloned from the high temperature microorganisms to produce recombinant expression vectors and microorganisms transformed with the same. Then, a high temperature-β-glycosidase and a high temperature-α-L-arabinofuranosidase are produced by enhancing the expression of an α-L-arabinofuranosidase derived from Thermotoga petrophila, which had a low expression level, and the optimum ratio of these two enzymes are determined. The present inventors have found that when the resultant is reacted with red ginseng extract, a large quantity of ginsenoside compound K is produced in a short time, resulting in a high yield, and thereby completed the present invention. Thus, an object of the present invention is to provide a composition for production of ginsenoside compound K comprising a high temperature-β-glycosidase and a high temperature-α-L-arabinofuranosidase.

In another aspect, an object of the present invention is to provide a preparation method for converting all the protopanaxadiol-type ginsenosides in red ginseng extract into ginsenoside compound K by using a high temperature-β-glycosidase and a high temperature-α-L-arabinofuranosidase.

### Solution to Problem

In one aspect, the present invention provides a composition for production of ginsenoside compound K comprising a high temperature-β-glycosidase and a high temperature-α-L-arabinofuranosidase.

In one aspect, the present invention may provide the use of a high temperature-β-glycosidase and a high temperature-α-L-arabinofuranosidase for production of ginsenoside compound K.

In one aspect of the present invention, the high temperature-β-glycosidase may be a β-glycosidase of Sulfolobus solfataricus, and the high temperature-αL-arabinofuranosidase may be an α-L-arabinofuranosidase of Thermotoga petrophila.

In one aspect of the present invention, the content of the high temperature-α-L-arabinofuranosidase may be 1 part by weight or more based on 100 parts by weight of the high temperature-β-glycosidase.

In one aspect of the present invention, the high temperature-α-L-arabinofuranosidase may be 2.5 parts by weight or more based on 100 parts by weight of the high temperature-β-glycosidase.

In one aspect of the present invention, the high temperature-β-glycosidase may be an enzyme consisting of the amino acid sequence of SEQ ID NO: 2, and the high temperature-α-L-arabinofuranosidase may be an enzyme consisting of the amino acid sequence of SEQ ID NO: 4.

In one aspect of the invention, the method may be a method for preparing a composition for production of ginsenoside compound K, comprising expression in E. coli transformed with a vector comprising the base sequence of SEQ ID NO: 3; and a vector comprising the base sequences of SEQ ID NO: 13 and SEQ ID NO: 14.

In another aspect, the present invention provides a method for preparing ginsenoside compound K, comprising the step of fermenting a saponin-containing material comprising at least one of ginsenoside Rb1, ginsenoside Rb2, ginsenoside Rc, and ginsenoside Rd with a high temperature-β-glycosidase and a high temperature-α-L-arabinofuranosidase.

In another aspect of the present invention, the step of fermentation may be fermentation using the composition for production of ginsenoside compound K according to any one of the aspects of the present invention.

In another aspect of the invention, the step of fermentation may be applying each of a high temperature-β-glycosidase and a high temperature-aα-L-arabinofuranosidase.

In another aspect of the present invention, the high temperature-β-glycosidase may be a β-glycosidase of Sulfolobus solfataricus, and the high temperature-α-L-arabinofuranosidase may be an α-L-arabinofuranosidase of Thermotoga petrophila.

In another aspect of the present invention, the high temperature-α-L-arabinofuranosidase may be applied in an amount of 1 part by weight or more based on 100 parts by weight of the high temperature-β-glycosidase.

In another aspect of the present invention, the saponin-containing material may be red ginseng extract.

In another aspect of the present invention, the fermentation may be fermentation at a temperature of 70° C. to 95° C.

In another aspect of the present invention, the fermentation may be fermentation at a temperature of 80° C. to 90° C.

### Advantageous Effects of Invention

The composition for production of ginsenoside compound K and the method for preparing ginsenoside compound K according to one aspect of the present invention allow high temperature-β-glycosidase and high temperature-α-L-arabinofuranosidase to exhibit stable activity even at high temperatures, thereby increasing a reaction rate.

The composition for production of ginsenoside compound K and the method for preparing ginsenoside compound K according to one aspect of the present invention allow a large quantity of ginsenoside compound K to be produced in a short time, thereby exhibiting an effect of producing a high yield, and thus can be utilized industrially.

### BRIEF DESCRIPTION OF DRAWINGS

FIG. 1 shows the results of Test Example 1 regarding α-L-arabinofuranosidases in cell debris, enzyme suspension and purified enzyme liquid when α-L-arabinofuranosidases derived from Thermotoga petrophila were expressed in various host strains and coexpressed with chaperone.

FIG. 2 shows the decrease of compound Mc when the concentration of α-L-arabinofuranosidase was varied while the concentration of high temperature-β-glycosidase was fixed at 2 mg/ml and red ginseng extract was used as a substrate.

FIG. 3 shows the production of ginsenoside compound K by 2 mg/ml of high temperature-β-glycosidase when red ginseng extract was used as a substrate.

FIG. 4 shows the production of ginsenoside compound K by 2 mg/ml of β-glycosidase and 0.05 mg/ml of α-L-arabinofuranosidase when red ginseng extract was used as a substrate.

### DESCRIPTION OF EMBODIMENTS

### Embodiments

Hereinafter, the present invention will be described in detail.

In one aspect, the present invention provides a composition for production of ginsenoside compound K comprising a high temperature-β-glycosidase and a high temperature-α-L-arabinofuranosidase.

As used herein, the term “high temperature” enzyme refers to an enzyme that exhibits optimum activity at a high temperature of 70-95° C., rather than an intermediate temperature of 10-50° C., which is the optimum temperature for enzyme activity.

In one aspect of the present invention, the high temperature-β-glycosidase may be a β-glycosidase of Sulfolobus solfataricus, and the high temperature-α-L-arabinofuranosidase may be an α-L-arabinofuranosidase of Thermotoga petrophila.

In one aspect of the present invention, the high temperature-β-glycosidase and the high temperature-α-L-arabinofuranosidase of the present invention are obtained from Sulfolobus solfataricus and Thermotoga petrophila, which are high temperature organisms, by 1) directly isolating them from these strains and purifying them or 2) cloning the genes of each of the enzymes from the strains, expressing them in a recombinant expression vector, and purifying them. The method for obtaining the enzymes from microorganisms is a conventional method in the art (Sambrook, J. and Russell, D. W. Molecular Cloning 3rd Ed. Cold Spring Harbor Laboratory, 2001).

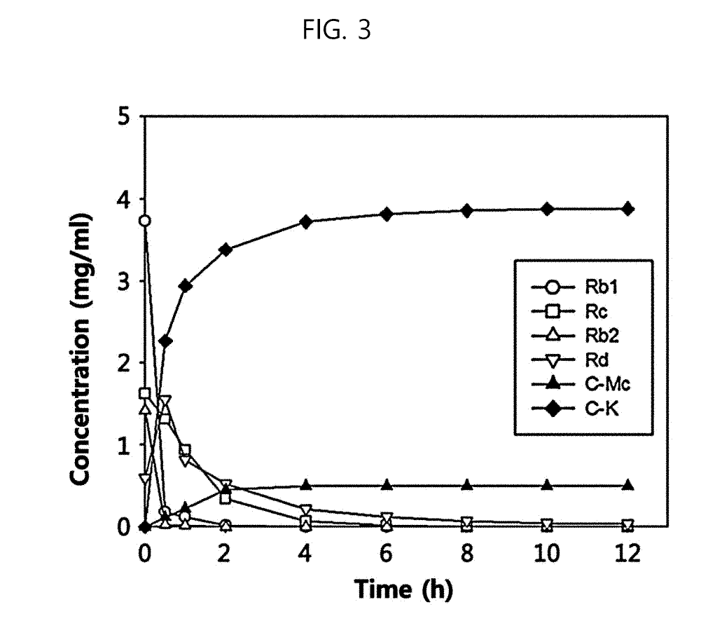

When the β-glycosidase obtained by a conventional method is applied to red ginseng extract, ginsenoside Rc and compound Mc among protopanaxadiol-type saponins are left, which limits the production yield of ginsenoside compound K (FIG. 3). Thus, in one aspect, the present invention provides a method for converting all the protopanaxadiol-type saponins in red ginseng extract or tiny-sized ginseng extract to compound K by applying α-L-arabinofuranosidase simultaneously.

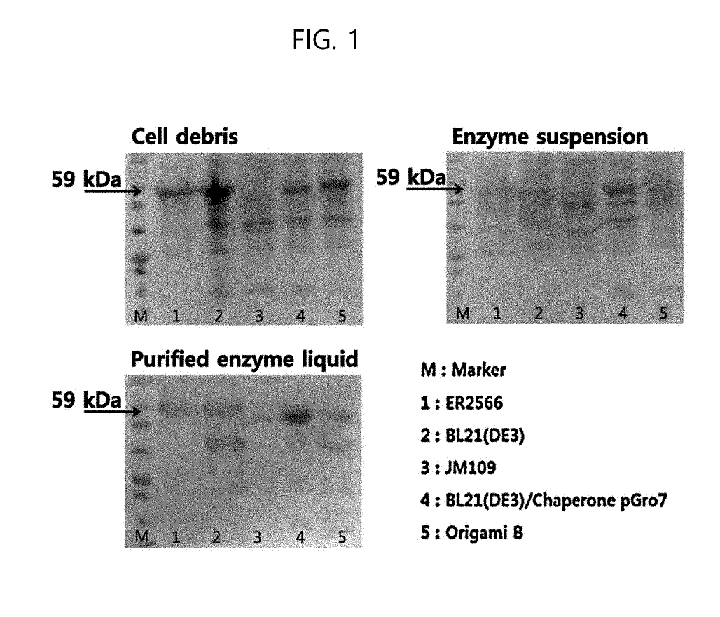

In one aspect of the present invention, α-L-arabinofuranosidase derived from Thermotoga petrophila exhibited about 17 times higher activity than α-L-arabinofuranosidase derived from Caldicellulosiruptor saccharolyticus, which has been conventionally used in the production of ginsenoside compound K. Also, its expression pattern was enhanced by host cell selection and the introduction of chaperone (FIG. 1).

In one aspect of the present invention, the content of the high temperature-α-L-arabinofuranosidase may be 1 part by weight or more based on 100 parts by weight of the high temperature-β-glycosidase.

Specifically, the content of the temperature-α-L-arabinofuranosidase may be 1 part by weight or more, 1.5 parts by weight or more, 2.0 parts by weight or more, 2.1 parts by weight or more, 2.2 parts by weight or more, 2.3 parts by weight or more, 2.4 parts by weight or more, 2.5 parts by weight or more, 2.6 parts by weight or more, 2.7 parts by weight or more, 2.8 parts by weight or more, or 3.0 parts by weight or more based on 100 parts by weight of the high temperature-β-glycosidase. Also, the content of the high temperature-α-L-arabinofuranosidase may be 5.0 parts by weight or less, 4.5 parts by weight or less, or 4.0 parts by weight or less based on 100 parts by weight of the high temperature-β-glycosidase.

The high temperature-α-L-arabinofuranosidase can achieve the maximum generation of compound K economically while minimizing the concentration of the enzyme, when the weight ratio of the high temperature-β-glycosidase is within the above range.

Preferably, the content of the temperature-α-L-arabinofuranosidase may be 2 parts by weight or more based on 100 parts by weight of the high temperature-β-glycosidase.

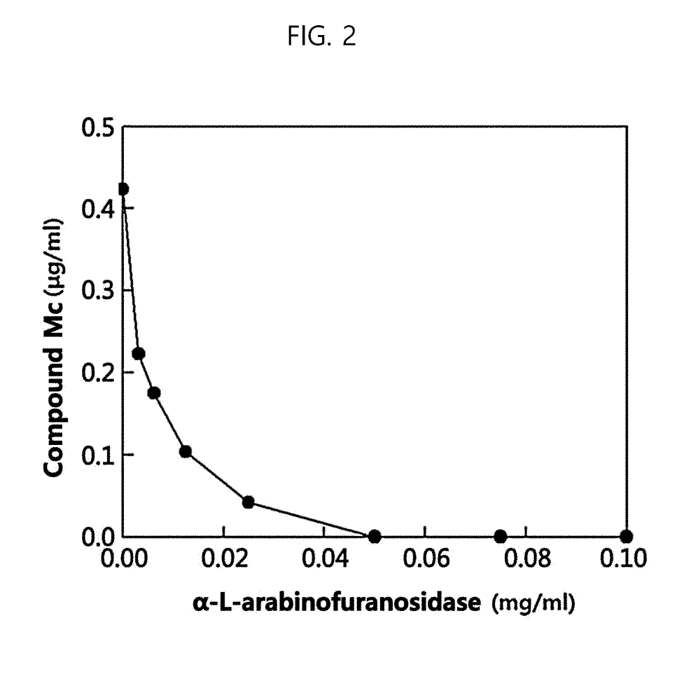

More preferably, the content of the high temperature-α-L-arabinofuranosidase may be 2.5 parts by weight or more based on 100 parts by weight of the high temperature-β-glycosidase. In one aspect of the present invention, when red ginseng extract is used as a substrate, all of the remaining compounds Mc are converted to compounds K at a concentration ratio of β-glycosidase derived from Thermotoga petrophila and α-L-arabinofuranosidase derived from Sulfolobus solfataricus of 40:1 (FIG. 2).

As described above, the composition for production of ginsenoside compound K comprising a high temperature-β-glycosidase and a high temperature-α-L-arabinofuranosidase according to one aspect of the present invention controls the reaction rate rapidly at a high temperature of 85° C. and thereby achieves the effect of producing ginsenoside compound K in a short time at a high yield and using a low enzyme concentration, when reacted with a mixture of ginsenosides Rb1, Rb2, Rc, and Rd, which are major diol-type saponins in red ginseng extract, in a mixed solution of a buffer solution and an aqueous solvent.

In one aspect of the present invention, the high temperature-β-glycosidase may be an enzyme consisting of the amino acid sequence of SEQ ID NO: 2, and the high temperature-α-L-arabinofuranosidase may be an enzyme consisting of the amino acid sequence of SEQ ID NO: 4.

In one aspect of the invention, the method may be a method for preparing a composition for production of ginsenoside compound K, comprising expression in E. coli transformed with a vector comprising the base sequence of SEQ ID NO: 3; and a vector comprising the base sequences of SEQ ID NO: 13 and SEQ ID NO: 14. The vector comprising the base sequences of SEQ ID NO: 13 and SEQ ID NO: 14 may be chaperone pGrp7.

In another aspect, the present invention provides a method for preparing ginsenoside compound K, comprising the step of fermenting a saponin-containing material comprising at least one of ginsenoside Rb1, ginsenoside Rb2, ginsenoside Rc, and ginsenoside Rd with a high temperature-β-glycosidase and a temperature-α-L-arabinofuranosidase.

In another aspect of the present invention, the step of fermentation may be fermentation using the composition for production of ginsenoside compound K according to any one of the aspects of the present invention.

In another aspect of the invention, the step of fermentation may be applying each of a high temperature-β-glycosidase and a high temperature-α-L-arabinofuranosidase. In another aspect of the present invention, the high temperature-β-glycosidase may be a β-glycosidase of Sulfolobus solfataricus, and the high temperature-α-L-arabinofuranosidase may be an α-L-arabinofuranosidase of Thermotoga petrophila. In another aspect of the present invention, the high temperature-α-L-arabinofuranosidase may be applied in an amount of 1 part by weight or more based on 100 parts by weight of the high temperature-β-glycosidase. Specifically, the content of the high temperature-α-L-arabinofuranosidase may be 1 part by weight or more, 1.5 parts by weight or more, 2.0 parts by weight or more, 2.1 parts by weight or more, 2.2 parts by weight or more, 2.3 parts by weight or more, 2.4 parts by weight or more, 2.5 parts by weight or more, 2.6 parts by weight or more, 2.7 parts by weight or more, 2.8 parts by weight or more, or 3.0 parts by weight or more based on 100 parts by weight of the high temperature-β-glycosidase. Also, the content of the high temperature-α-L-arabinofuranosidase may be 5.0 parts by weight or less, 4.5 parts by weight or less, or 4.0 parts by weight or less based on 100 parts by weight of the high temperature-β-glycosidase.

In one embodiment of the present invention, a) PCR is performed with genomic DNA of Sulfolobus solfataricus and Thermotoga petrophila and their respective primers to amplify the DNA fragments comprising each of high temperature-β-glycosidase and high temperature-α-L-arabinofuranosidase genes; b) the amplified DNA fragments comprising each of high temperature-β-glycosidase and high temperature-α-L-arabinofuranosidase gene are treated with restriction enzymes and each of them is cloned into plasmid vectors pET-24a(+) and pET-21a(+) to construct recombinant expression vectors pET-24a(+)/β-glycosidase and pET-21a(+)/α-L-arabinofuranosidase; c) E. coli ER2566 is transformed with the vectors according to a conventional transformation method; d) E. coli transformed with each of high temperature-β-glycosidase genes and high temperature α-L-arabinofuranosidase genes is cultured; e) gene expression is induced during culture to produce a high temperature-β-glycosidase and a high temperature-α-L-arabinofuranosidase; and f) the expressed high temperature-β-glycosidase and high temperature-α-L-arabinofuranosidase proteins are isolated and obtained.

The pET-21a(+)/α-L-arabinofuranosidase in the above step c) may be transformed together with the chaperone vector pGro7 into BL21(DE3), which shows the highest expression among various strains such as E. coli ER2566, BL21(DE3), JM109 and Origami B, as a host.

The process of isolating the high temperature (β-glucosidase and high temperature-α-L-arabinofuranosidase proteins expressed in the above step f) may consist of the steps of: (a) lysing the culture solution of microorganisms; (b) centrifuging the cell lysate to obtain a supernatant; (c) subjecting the supernatant to heat treatment at a high temperature and centrifuging the resultant; and (d) filtering the thus-obtained supernatant to isolate an enzyme liquid.

In the above step (a), preferably, cells are lysed at a pressure of about 15,000 lb/in² using a device such as a French press. In the above step (c), preferably, the cell supernatant is subjected to heat treatment at a temperature of 75° C. for about 10 minutes. In the above step (d), preferably, the filtration is performed using a filter paper of about 0.45 μm.

Also, the substrate may be ginsenosides Rb1, Rb2, Rc, and Rd, which are diol-type saponins in red ginseng extract, and may be used as a mixture in the preparation of ginsenoside compound K. The reaction solvent may be a buffer solution such as Mcllvaine buffer.

As described above, the reaction between the high temperature-β-glycosidase and high temperature-α-L-arabinofuranosidase and the substrate in the reaction solvent is performed preferably at a pH of 5.0 to 7.0 and a temperature of 70 to 95° C., more preferably at a pH of 6.0 and a temperature of 85° C.

The method for preparing ginsenoside compound K using a high temperature-β-glycosidase and a high temperature-α-L-arabinofuranosidase according to the present invention allows a high temperature-β-glycosidase derived from Sulfolobus solfataricus and a high temperature-α-L-arabinofuranosidase derived from Thermotoga petrophila to exhibit stable activity even at high temperatures, thereby increasing a reaction rate. As a result, it allows a large quantity of ginsenoside compound k to be produced in a short time, thereby exhibiting an effect of producing a high yield, and thus can be utilized industrially.

In another aspect of the present invention, the saponin-containing material may be red ginseng extract.

In another aspect of the present invention, the fermentation may be performed at a temperature of 70° C. to 95° C. Specifically, the fermentation temperature may be 70° C. or more, 72° C. or more, 74° C. or more, 76° C. or more, 78° C. or more, 80° C. or more, 82° C. or more, or 84° C. or more. Also, the fermentation temperature may be 95° C. or less, 93° C. or less, 91° C. or less, 90° C. or less, 88° C. or less, 86° C. or less, or 84° C. or less. When the temperature is within the above range, the production yield of ginsenoside K is excellent.

Hereinafter, preferred examples of the present invention will be described to facilitate understanding of the present invention. However, the following examples are provided only to facilitate understanding of the present invention, and the scope of the present invention is not limited thereto.

### Example 1

Preparation of a Recombinant Expression Vector Comprising a High Temperature-α-glycosidase Coding Base Sequence or a High Temperature-α-L-arabinofuranosidase Coding Base Sequence, and a Transformed Microorganism

In order to prepare a high temperature-β-glycosidase, a β-glycosidase gene derived from Sulfolobus solfataricus was isolated. Also, in order to prepare a high temperature-α-L-arabinofuranosidase, an α-L-arabinofuranosidase gene derived from Thermotoga petrophila was isolated.

Specifically, Sulfolobus solfataricus and Thermotoga petrophila, whose base sequence and amino acid sequence are already specified, were selected and the genomic DNA of each was extracted. The Sulfolobus solfataricus used was DSM 1617 purchased from the DSMZ (Germany), and the Thermotoga petrophila used was DSM 13995 purchased from the DSMZ (Germany).

Also, primers were prepared using the base sequence of the β-glycosidase gene of Sulfolobus solfataricus (GenBank Accession No. M34696) and the base sequence of the α-L-arabinofuranosidase gene of Thermotoga petrophila (GenBank Accession No. ABQ46651, respectively.

The DNA base sequence of the β-glycosidase of Sulfolobus solfataricus was as shown in SEQ ID NO: 1, and the amino acid sequence thereof was as shown in SEQ ID NO: 2.

The DNA base sequence of the α-L-arabinofuranosidase of Thermotoga petrophila was as shown in SEQ ID NO: 3, and the amino acid sequence thereof was as shown in SEQ ID NO: 4.

The forward and reverse primers for the β-glycosidase of Sulfolobus solfataricus were as shown in SEQ ID NO: 5 and SEQ ID NO: 6, respectively.

In addition, the forward and reverse primers for α-L-arabinofuranosidase of Thermotoga petrophila were as shown in SEQ ID NO: 7 and SEQ ID NO: 8, respectively.

Polymerase chain reaction (PCR) was performed using the genomic DNA and primers to amplify the base sequences of the corresponding genes. After the respective genes were obtained in large quantities by the above procedure, they were inserted into plasmid vectors pET-24a(+) and pET-21a to prepare recombinant expression vectors pET-24a(+)/β-glycosidase and pET-21a/α-L-arabinofuranosidase.

The plasmid vector pET-24a(+) was as shown in SEQ ID NO: 9.

The plasmid vector pET-21a was as shown in SEQ ID NO: 10.

The recombinant expression vector pET-24a(+)/β-glycosidase was as shown in SEQ ID NO: 11.

The recombinant expression vector pET-21a/α-L-arabinofuranosidase was as shown in SEQ ID NO: 12.

Also, the thus-prepared recombinant expression vectors were transformed into E. coli strain ER2566 by a conventional transformation method. pET-21a/α-L-arabinofuranosidase was also transformed into E. coli strains BL21(DE3), JM109 and Origami B.

The E. coli strains ER2566 and BL21(DE3) were purchased from New England Biolabs (NEB).

The E. coli strain JM109 was purchased from Takara.

The E. coli strain Origami B was purchased from Novagen.

BL21(DE3), which among them exhibited the highest expression, as a host was transformed with pET-21a/α-L-arabinofuranosidase and the chaperone vector pGro7, which was a commercial chaperone vector purchased from Takara. The chaperone vector pGro7, which was an independent plasmid, was co-transformed with the pET-21a/α-L-arabinofuranosidase vector into the strain BL21(DE3).

The chaperone vector pGro7 was a vector that simultaneously expresses GroEL and GroES genes. The GroEL gene was as shown in SEQ ID NO: 13, and the GroES gene was as shown in SEQ ID NO: 14. A schematic diagram of the chaperon pGro7 vector is shown in FIG. 5.

The transformed recombinant E. coli is referred to as E. coli strain ER2566 pET-24a(+)/β-glycosidase, E. coli strains ER2566, BL21(DE3), JM109, and Origami B pET-21a/α-L-arabinofuranosidase, and E. coli strain BL21(DE3) pET-21a/α-L-arabinofuranosidase-pGro7.

The transformed E. coli was added with 20% glycerine solution and stored frozen before culture.

### Example 2

### Expression and Purification of a High Temperature-β-Glycosidase and a High Temperature-α-L-Arabinofuranosidase

In order to mass produce β-glycosidase and α-L-arabinofuranosidase, the frozen E. coli strain ER2566 pET-24a(+)/β-glycosidase, E. coli strains ER2566, BL21(DE3), JM109, and Origami B pET-21a/α-L-arabinofuranosidase, and E. coli strain BL21(DE3) pET-21a/α-L-arabinofuranosidase-pGro7 each were seeded into a 250 ml flask containing 50 ml of LB medium, and then subjected to shaking culture in a shaking incubator at 37° C. until the absorbance at 600 nm reached 2.0. Then, the culture solution was added to a 21 Erlenmeyer flask containing 500 ml of LB medium and cultured until the absorbance at 600 nm reached 0.8. During the process, the stirring speed was 200 rpm and the culture temperature was 37° C. The resultant was added with 0.1 mM IPTG (isopropyl-beta-thiogalactoside) to induce production of the overexpressed enzyme. The stirring speed was adjusted to 150 rpm and the culture temperature was adjusted to 16° C.

In order to purify the thus-obtained high temperature-β-glycosidase and high temperature-α-L-arabinofuranosidase, the cultures of the transformed strains were centrifuged at 4,000×g for 4 to 30 minutes. Then, the cell solutions were lysed using a French press at 15,000 lb/in². The cell lysates were centrifuged again at 13,000×g for 4 to 20 minutes and subjected to heat treatment at a high temperature of 75° C. for 10 minutes. The thus-obtained heat-treated product was centrifuged again at 13,000×g for 4 to 20 minutes. The resultant supernatant was filtered with a 0.45 μm filter paper and isolated as an enzyme liquid which can be used for the production of ginsenoside compound K.

### Test Example 1

### Determination of the Expression Level of α-L-Arabinofuranosidase

The expression levels of the α-L-arabinofuranosidase enzyme liquids isolated from various host strains, the enzyme suspensions before subjected to heat treatment, and the cell debris obtained by centrifugation according to Example 2 were qualitatively compared through SDS-PAGE analysis.

As a result, as shown in FIG. 1, it was found from the cell debris that α-L-arabinofuranosidase expressed in the E. coli strain BL21(DE3) (well No. 2) was most expressed. Also, it was found from the purified enzyme liquid and the enzyme suspension that in the case of coexpression using chaperone pGro7 in the E. coli strain BL21(DE3) (well No. 4), α-L-arabinofuranosidase reached the highest concentration, and the expression of α-L-arabinofuranosidase of relatively high solubility was enhanced.

### Test Example 2

### Experiment on the Optimum Ratio of High Temperature-α-Glycosidase and High Temperature-α-L-Arabinofuranosidase

It was found that when the high temperature-β-glycosidase isolated in Example 2 was applied to red ginseng extract, ginsenoside Rc and compound Mc among protopanaxadiol-type saponins were left, which limited the production yield of ginsenoside compound K (FIG. 3).

In order to convert the residual ginsenoside Rc and compound Mc into compound K, α-L-arabinofuranosidase was added for co-treatment with β-glycosidase, and then the compound K production was compared.

The high temperature-β-glycosidase isolated in Example 2 was added with varying concentration of α-L-arabinofuranosidase, which was confirmed to have enhanced expression in Test Example 1, and the optimum ratio of the enzymes was determined in the following manner. The two enzymes were reacted with red ginseng extract and compared for the degree of compound K production.

In order to determine the optimum concentration ratio of β-glycosidase and α-L-arabinofuranosidase, red ginseng extract containing about 7.5 mg/ml of protopanaxadiol-type saponins, 50 mM Mcilvaine buffer solution (pH 6.0), and a mixture of the two enzymes were applied.

When 2 mg/ml of β-glycosidase alone was applied to red ginseng extract as a substrate, it was found that most of ginsenosides Rd disappeared after 12 hours as shown in FIG. 3.

The concentration of α-L-arabinofuranosidase at which all of compounds Mc (C-Mc) are converted was determined by varying the concentration of α-L-arabinofuranosidase with the concentration of β-glycosidase fixed at 2 mg/ml. Specifically, the concentration of α-L-arabinofuranosidase was decreased from 0.1 mg/ml to 0.0032 mg/ml. As a result, as shown in FIG. 2, it was found that when α-L-arabinofuranosidase at a concentration of 0.05 mg/ml or more was applied with the concentration of β-glycosidase fixed at 2 mg/ml, all of the compounds Mc were converted.

### Example 3

Production of Ginsenoside Compound K using High Temperature-β-Glycosidase and High Temperature α-L-Arabinofuranosidase

In order to develop a method for preparing ginsenoside compound K using the high temperature-β-glycosidase of Example 2 and the α-L-arabinofuranosidase with enhanced expression in Test Example 1, the production of ginsenoside compound K over time was measured using red ginseng extract and tiny-sized ginseng extract at an optimum ratio of the enzymes in each substrate as determined above.

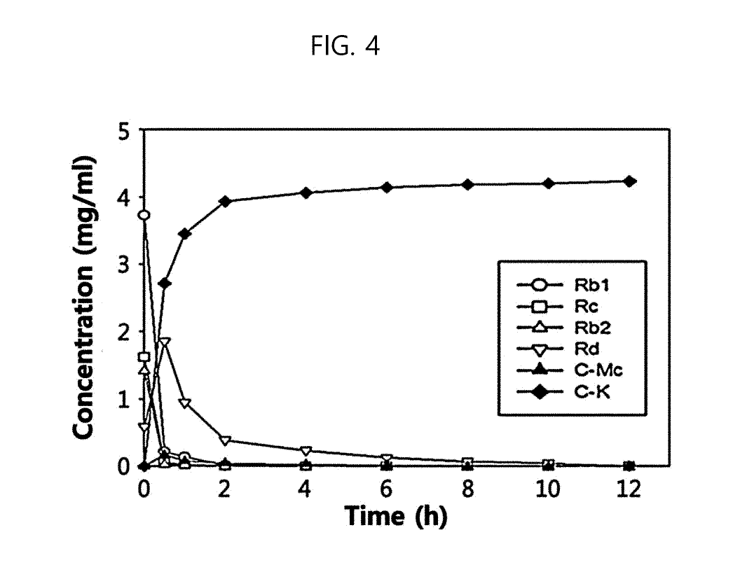

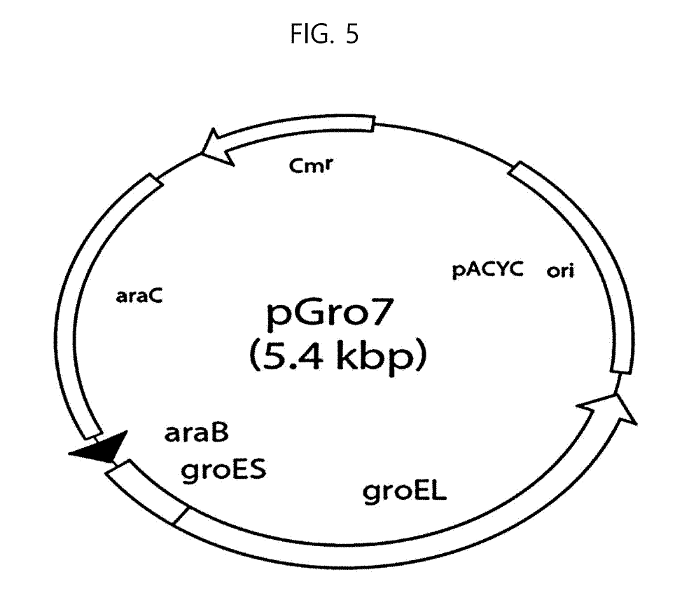

The test results are shown in FIG. 4. FIG. 4 is a graph showing the production of ginsenoside compound K by 2.0 mg/ml of β-glycosidase and 0.05 mg/ml of α-L-arabinofuranosidase of the present invention in red ginseng extract containing about 7.5 mg/ml of protopanaxadiol-type saponins as a substrate. FIG. 4 shows that after 12 hours, all of the materials were converted to produce 4.2 mg/ml of ginsenoside compound K (C-K).

Until now, a suspension of β-glycosidase (2.3 mg/ml) from Sulfolobus solfataricus and α-L-arabinofuranosidase (0.39 mg/ml) from Thermotoga petrophila has been found to achieve the highest productivity in production of ginsenoside compound K. It has been reported that the use of the suspension in red ginseng extract containing about 7.5 mg/ml of protopanaxadiol-type saponins resulted in production of 4.2 mg/ml of ginsenoside compound K for 12 hours (Kyung-Chul Shin et al. 2015, Compound K Production from Red Ginseng Extract by β-Glycosidase from Sulfolobus solfataricus Supplemented with α-L-arabinofuranosidase from Caldicellulosiruptor saccharolyticus. PLoS One. 28;10(12):e0145876.).

Upon comparing the above case and the present invention, in the case of using the high temperature-β-glycosidase and the high temperature-α-L-arabinofuranosidase according to one aspect of the present invention, the total enzyme concentration was about 1.3 times lower than in the case of using the two enzymes, and the concentration of α-L-arabinofuranosidase among them was 8 times lower than the above case, and the productivity increased by about 1.2 times. Thus, it was confirmed that the productivity per enzyme concentration in this experiment was 1.3 times higher than the above case.

Sequence Listing Free Text

SEQ ID NO: 1

| | |
|---|---|
| ggatcaatac taggaggagt agcatataat tacgttacac aattttataa cccaatatat | 60 |
| tcaatagacc ttatgcttat cctatcctct attctaagat tctcggtatc tcccctattc | 120 |
| ttgaccataa aagatactcg ctcaaagctt aaataatatt aatcataaat aaagtcatgt | 180 |
| actcatttcc aaatagcttt aggtttggtt ggtcccaggc cggatttcaa tcagaaatgg | 240 |
| gaacaccagg gtcagaagat ccaaatactg actggtataa atgggttcat gatccagaaa | 300 |
| acatggcagc gggattagta agtggagatc taccagaaaa tgggccaggc tactggggaa | 360 |
| actataagac atttcacgat aatgcacaaa aaatgggatt aaaaatagct agactaaatg | 420 |
| tggaatggtc taggatattt cctaatccat taccaaggcc acaaaacttt gatgaatcaa | 480 |
| aacaagatgt gacagaggtt gagataaacg aaaacgagtt aaagagactt gacgagtacg | 540 |
| ctaataaaga cgcattaaac cattacaggg aaatattcaa ggatcttaaa agtagaggac | 600 |
| tttactttat actaaacatg tatcattggc cattacctct atggttacac gacccaataa | 660 |
| gagtaagaag aggagatttt actggaccaa gtggttggct aagtactaga acagtttacg | 720 |
| aattcgctag attctcagct tatatagctt ggaaattcga tgatctagtg gatgagtact | 780 |
| caacaatgaa tgaacctaac gttgttggag gtttaggata cgttggtgtt aagtccggtt | 840 |
| ttcccccagg atacctaagc tttgaacttt cccgtagggc aatgtataac atcattcaag | 900 |
| ctcacgcaag agcgtatgat gggataaaga gtgtttctaa aaaaccagtt ggaattattt | 960 |
| acgctaatag ctcattccag ccgttaacgg ataaagatat ggaagcggta gagatggctg | 1020 |
| aaaatgataa tagatggtgg ttctttgatg ctataataag aggtgagatc accagaggaa | 1080 |
| acgagaagat tgtaagagat gacctaaagg gtagattgga ttggattgga gttaattatt | 1140 |
| acactaggac tgttgtgaag aggactgaaa agggatacgt tagcttagga ggttacggtc | 1200 |
| acggatgtga gaggaattct gtaagtttag cgggattacc aaccagcgac ttcggctggg | 1260 |
| agttcttccc agaaggttta tatgacgttt tgacgaaata ctggaataga tatcatctct | 1320 |
| atatgtacgt tactgaaaat ggtattgcgg atgatgccga ttatcaaagg ccctattatt | 1380 |
| tagtatctca cgtttatcaa gttcatagag caataaatag tggtgcagat gttagagggt | 1440 |
| atttacattg gtctctagct gataattacg aatgggcttc aggattctct atgaggtttg | 1500 |
| gtctgttaaa ggtcgattac aacactaaga gactatactg gagaccctca gcactagtat | 1560 |
| atagggaaat cgccacaaat ggcgcaataa ctgatgaaat agagcactta aatagcgtac | 1620 |
| ctccagtaaa gccattaagg cactaaactt tctcaagtct cactatacca aatgagtttt | 1680 |
| cttttaatct tattctaatc tcattttcat tagattgcaa tactttcata ccttctatat | 1740 |
| tatttatttt gtaccttttg ggatc | 1765 |

SEQ ID NO: 2
Met Tyr Ser Phe Pro Asn Ser Phe Arg Phe Gly Trp Ser Gln Ala Gly
Phe Gln Ser Glu Met Gly Thr Pro Gly Ser Glu Asp Pro Asn Thr Asp
Trp Tyr Lys Trp Val His Asp Pro Glu Asn Met Ala Ala Gly Leu Val
Ser Gly Asp Leu Pro Glu Asn Gly Pro Gly Tyr Trp Gly Asn Tyr Lys
Thr Phe His Asp Asn Ala Gln Lys Met Gly Leu Lys Ile Ala Arg Leu
Asn Val Glu Trp Ser Arg Ile Phe Pro Asn Pro Leu Pro Arg Pro Gln
Asn Phe Asp Glu Ser Lys Gln Asp Val Thr Glu Val Glu Ile Asn Glu
Asn Glu Leu Lys Arg Leu Asp Glu Tyr Ala Asn Lys Asp Ala Leu Asn
His Tyr Arg Glu Ile Phe Lys Asp Leu Lys Ser Arg Gly Leu Tyr Phe
Ile Leu Asn Met Tyr His Trp Pro Leu Pro Leu Trp Leu His Asp Pro
Ile Arg Val Arg Arg Gly Asp Phe Thr Gly Pro Ser Gly Trp Leu Ser
Thr Arg Thr Val Tyr Glu Phe Ala Arg Phe Ser Ala Tyr Ile Ala Trp
Lys Phe Asp Asp Leu Val Asp Glu Tyr Ser Thr Met Asn Glu Pro Asn
Val Val Gly Gly Leu Gly Tyr Val Gly Val Lys Ser Gly Phe Pro Pro
Gly Tyr Leu Ser Phe Glu Leu Ser Arg Arg Ala Met Tyr Asn Ile Ile
Gln Ala His Ala Arg Ala Tyr Asp Gly Ile Lys Ser Val Ser Lys Lys
Pro Val Gly Ile Ile Tyr Ala Asn Ser Ser Phe Gln Pro Leu Thr Asp
Lys Asp Met Glu Ala Val Glu Met Ala Glu Asn Asp Asn Arg Trp Trp
Phe Phe Asp Ala Ile Ile Arg Gly Glu Ile Thr Arg Gly Asn Glu Lys
Ile Val Arg Asp Asp Leu Lys Gly Arg Leu Asp Trp Ile Gly Val Asn
Tyr Tyr Thr Arg Thr Val Val Lys Arg Thr Glu Lys Gly Tyr Val Ser
Leu Gly Gly Tyr Gly His Gly Cys Glu Arg Asn Ser Val Ser Leu Ala
Gly Leu Pro Thr Ser Asp Phe Gly Trp Glu Phe Phe Pro Glu Gly Leu
Tyr Asp Val Leu Thr Lys Tyr Trp Asn Arg Tyr His Leu Tyr Met Tyr
Val Thr Glu Asn Gly Ile Ala Asp Asp Ala Asp Tyr Gln Arg Pro Tyr
Tyr Leu Val Ser His Val Tyr Gln Val His Arg Ala Ile Asn Ser Gly
Ala Asp Val Arg Gly Tyr Leu His Trp Ser Leu Ala Asp Asn Tyr Glu
Trp Ala Ser Gly Phe Ser Met Arg Phe Gly Leu Leu Lys Val Asp Tyr
Asn Thr Lys Arg Leu Tyr Trp Arg Pro Ser Ala Leu Val Tyr Arg Glu
Ile Ala Thr Asn Gly Ala Ile Thr Asp Glu Ile Glu His Leu Asn Ser
Val Pro Pro Val Lys Pro Leu Arg His
SEQ ID NO: 3

| | |
|---|---|
| atgtcctaca ggatagtggt tgatccaaaa aaagttgtca agccgattag tagacacatc | 60 |
| tacggtcatt tcacggaaca tctgggaagg tgtatctacg gcggaattta tgaagaaggt | 120 |
| tctccgctct ccgatgaaag gggtttcaga aaggacgttc tggaggctgt aaagaggata | 180 |
| aaagttccga acttgagatg gcccggtgga aactttgtgt cgaactacca ctgggaagac | 240 |
| ggaataggtc ccaaagatca gaggcctgtc aggttcgatc tcgcctggca acaggaagag | 300 |
| acgaatagat ttggaacgga cgaattcatt gagtactgtc gtgagatagg agcagaacct | 360 |
| tacatcagta taaacatggg aactggaaca ctcgacgaag ctctccactg gcttgaatac | 420 |
| tgcaatggaa agggtaatac ctactacgct caactcagaa gaaagtacgg tcatccagaa | 480 |
| ccttacaacg taaagttctg gggaataggc aacgagatgt acggggaatg gcaggtaggc | 540 |
| cacatgacgg cggacgaata cgcaagagcc gccaaagaat acacgaaatg gatgaaggtt | 600 |
| ttcgatccta caattaaagc gatcgccgtg ggctgtgacg accctatatg gaatctcagg | 660 |
| gttcttcaag aagcaggtga tgtgattgac ttcatatcct accatttcta cacagggtcc | 720 |
| gaggattact acgaaacagt ttccacggtt taccttctca aagaaagact catcggagtg | 780 |
| aaaaagctca ttgatatggt ggatactgct agaaagagag gtgtcaaaat cgcccttgat | 840 |
| gaatggaacg tatggtacag agtgtccgat aacaagctcg aagaacctta cgatctcaaa | 900 |
| gatggtatct ttgcatgtgg agtgcttgta cttcttcaaa agatgagcga catagtccca | 960 |
| cttgccaatc tcgcacagct tgtaaacgcc cttggagcta tacacaccga gaaagacggt | 1020 |
| ctcattctca cacccgttta caaggctttt gaactcatcg tgaatcattc cggagaaaag | 1080 |
| cttgtcaaga cccatgttga atcggagact tacaacatag aaggagtcat gttcatcaac | 1140 |
| aaaatgcctt tctctgtcga gaacgcaccg ttccttgatg ccgccgcttc catctcagaa | 1200 |
| gatggcaaga aacttttcat cgctgttgta aactacagga aagaagacgc tttgaaggtt | 1260 |
| ccaatcagag tggaaggtct gggacagaaa aaagccaccg tttatacact cacaggtccg | 1320 |
| gacgtgaacg cgagaaacac catggaaaat ccgaacgtcg ttgatattac ctccgaaacc | 1380 |
| atcaccgttg acaccgaatt tgaacacacg tttaaaccat tctcttgcag tgtgattgag | 1440 |
| gtagaattgg agtaa | 1455 |

SEQ ID NO: 4
Met Ser Tyr Arg Ile Val Val Asp Pro Lys Lys Val Val Lys Pro Ile
Ser Arg His Ile Tyr Gly His Phe Thr Glu His Leu Gly Arg Cys Ile
Tyr Gly Gly Ile Tyr Glu Glu Gly Ser Pro Leu Ser Asp Glu Arg Gly
Phe Arg Lys Asp Val Leu Glu Ala Val Lys Arg Ile Lys Val Pro Asn
Leu Arg Trp Pro Gly Gly Asn Phe Val Ser Asn Tyr His Trp Glu Asp
Gly Ile Gly Pro Lys Asp Gln Arg Pro Val Arg Phe Asp Leu Ala Trp
Gln Gln Glu Glu Thr Asn Arg Phe Gly Thr Asp Glu Phe Ile Glu Tyr
Cys Arg Glu Ile Gly Ala Glu Pro Tyr Ile Ser Ile Asn Met Gly Thr
Gly Thr Leu Asp Glu Ala Leu His Trp Leu Glu Tyr Cys Asn Gly Lys
Gly Asn Thr Tyr Tyr Ala Gln Leu Arg Arg Lys Tyr Gly His Pro Glu
Pro Tyr Asn Val Lys Phe Trp Gly Ile Gly Asn Glu Met Tyr Gly Glu
Trp Gln Val Gly His Met Thr Ala Asp Glu Tyr Ala Arg Ala Ala Lys
Glu Tyr Thr Lys Trp Met Lys Val Phe Asp Pro Thr Ile Lys Ala Ile
Ala Val Gly Cys Asp Asp Pro Ile Trp Asn Leu Arg Val Leu Gln Glu
Ala Gly Asp Val Ile Asp Phe Ile Ser Tyr His Phe Tyr Thr Gly Ser
Glu Asp Tyr Tyr Glu Thr Val Ser Thr Val Tyr Leu Leu Lys Glu Arg
Leu Ile Gly Val Lys Lys Leu Ile Asp Met Val Asp Thr Ala Arg Lys
Arg Gly Val Lys Ile Ala Leu Asp Glu Trp Asn Val Trp Tyr Arg Val
Ser Asp Asn Lys Leu Glu Glu Pro Tyr Asp Leu Lys Asp Gly Ile Phe
Ala Cys Gly Val Leu Val Leu Leu Gln Lys Met Ser Asp Ile Val Pro
Leu Ala Asn Leu Ala Gln Leu Val Asn Ala Leu Gly Ala Ile His Thr
Glu Lys Asp Gly Leu Ile Leu Thr Pro Val Tyr Lys Ala Phe Glu Leu
Ile Val Asn His Ser Gly Glu Lys Leu Val Lys Thr His Val Glu Ser
Glu Thr Tyr Asn Ile Glu Gly Val Met Phe Ile Asn Lys Met Pro Phe
Ser Val Glu Asn Ala Pro Phe Leu Asp Ala Ala Ala Ser Ile Ser Glu
Asp Gly Lys Lys Leu Phe Ile Ala Val Val Asn Tyr Arg Lys Glu Asp
Ala Leu Lys Val Pro Ile Arg Val Glu Gly Leu Gly Gln Lys Lys Ala
Thr Val Tyr Thr Leu Thr Gly Pro Asp Val Asn Ala Arg Asn Thr Met
Glu Asn Pro Asn Val Val Asp Ile Thr Ser Glu Thr Ile Thr Val Asp
Thr Glu Phe Glu His Thr Phe Lys Pro Phe Ser Cys Ser Val Ile Glu
Val Glu Leu Glu
SEQ ID NO: 5

| | |
|---|---|
| catatgtact catttccaaa tagc | 24 |

SEQ ID NO: 6

| | |
|---|---|
| ctcgagttag tgccttaatg gctttac | 27 |

SEQ ID NO: 7

| | |
|---|---|
| catatgatgt cctacaggat agtggttgat c | 31 |

SEQ ID NO: 8

| | |
|---|---|
| ctcgagctcc aattctacct caatcac | 27 |

SEQ ID NO: 9

| | |
|---|---|
| atccggatat agttcctcct ttcagcaaaa aacccctcaa gacccgttta gaggccccaa | 60 |
| ggggttatgc tagttattgc tcagcggtgg cagcagccaa ctcagcttcc tttcgggctt | 120 |
| tgttagcagc cggatctcag tggtggtggt ggtggtgctc gagtgcggcc gcaagcttgt | 180 |
| cgacggagct cgaattcgga tccgcgaccc atttgctgtc caccagtcat gctagccata | 240 |
| tgtatatctc cttcttaaag ttaaacaaaa ttatttctag aggggaattg ttatccgctc | 300 |
| acaattcccc tatagtgagt cgtattaatt tcgcgggatc gagatctcga tcctctacgc | 360 |
| cggacgcatc gtggccggca tcaccggcgc cacaggtgcg gttgctggcg cctatatcgc | 420 |
| cgacatcacc gatggggaag atcgggctcg ccacttcggg ctcatgagcg cttgtttcgg | 480 |
| cgtgggtatg gtggcaggcc ccgtggccgg gggactgttg ggcgccatct ccttgcatgc | 540 |
| accattcctt gcggcggcgg tgctcaacgg cctcaaccta ctactgggct gcttcctaat | 600 |
| gcaggagtcg cataagggag agcgtcgaga tcccggacac catcgaatgg cgcaaaacct | 660 |
| ttcgcggtat ggcatgatag cgcccggaag agagtcaatt cagggtggtg aatgtgaaac | 720 |
| cagtaacgtt atacgatgtc gcagagtatg ccggtgtctc ttatcagacc gtttcccgcg | 780 |
| tggtgaacca ggccagccac gtttctgcga aaacgcggga aaaagtggaa gcggcgatgg | 840 |
| cggagctgaa ttacattccc aaccgcgtgg cacaacaact ggcgggcaaa cagtcgttgc | 900 |
| tgattggcgt tgccacctcc agtctggccc tgcacgcgcc gtcgcaaatt gtcgcggcga | 960 |
| ttaaatctcg cgccgatcaa ctgggtgcca gcgtggtggt gtcgatggta gaacgaagcg | 1020 |
| gcgtcgaagc ctgtaaagcg gcggtgcaca atcttctcgc gcaacgcgtc agtgggctga | 1080 |
| tcattaacta tccgctggat gaccaggatg ccattgctgt ggaagctgcc tgcactaatg | 1140 |
| ttccggcgtt atttcttgat gtctctgacc agacacccat caacagtatt attttctccc | 1200 |
| atgaagacgg tacgcgactg ggcgtggagc atctggtcgc attgggtcac cagcaaatcg | 1260 |
| cgctgttagc gggcccatta agttctgtct cggcgcgtct gcgtctggct ggctggcata | 1320 |
| aatatctcac tcgcaatcaa attcagccga tagcggaacg ggaaggcgac tggagtgcca | 1380 |
| tgtccggttt tcaacaaacc atgcaaatgc tgaatgaggg catcgttccc actgcgatgc | 1440 |
| tggttgccaa cgatcagatg gcgctgggcg caatgcgcgc cattaccgag tccgggctgc | 1500 |
| gcgttggtgc ggatatctcg gtagtgggat acgacgatac cgaagacagc tcatgttata | 1560 |
| tcccgccgtt aaccaccatc aaacaggatt ttcgcctgct ggggcaaacc agcgtggacc | 1620 |
| gcttgctgca actctctcag ggccaggcgg tgaagggcaa tcagctgttg cccgtctcac | 1680 |
| tggtgaaaag aaaaaccacc ctggcgccca atacgcaaac cgcctctccc cgcgcgttgg | 1740 |
| ccgattcatt aatgcagctg gcacgacagg tttcccgact ggaaagcggg cagtgagcgc | 1800 |
| aacgcaatta atgtaagtta gctcactcat taggcaccgg gatctcgacc gatgcccttg | 1860 |
| agagccttca acccagtcag ctccttccgg tgggcgcggg gcatgactat cgtcgccgca | 1920 |
| cttatgactg tcttctttat catgcaactc gtaggacagg tgccggcagc gctctgggtc | 1980 |
| attttcggcg aggaccgctt tcgctggagc gcgacgatga tcggcctgtc gcttgcggta | 2040 |
| ttcggaatct tgcacgccct cgctcaagcc ttcgtcactg gtcccgccac caaacgtttc | 2100 |
| ggcgagaagc aggccattat cgccggcatg gcggccccac gggtgcgcat gatcgtgctc | 2160 |
| ctgtcgttga ggacccggct aggctggcgg ggttgcctta ctggttagca gaatgaatca | 2220 |
| ccgatacgcg agcgaacgtg aagcgactgc tgctgcaaaa cgtctgcgac ctgagcaaca | 2280 |
| acatgaatgg tcttcggttt ccgtgtttcg taaagtctgg aaacgcggaa gtcagcgccc | 2340 |
| tgcaccatta tgttccggat ctgcatcgca ggatgctgct ggctaccctg tggaacacct | 2400 |
| acatctgtat taacgaagcg ctggcattga ccctgagtga tttttctctg gtcccgccgc | 2460 |
| atccataccg ccagttgttt accctcacaa cgttccagta accgggcatg ttcatcatca | 2520 |
| gtaacccgta tcgtgagcat cctctctcgt ttcatcggta tcattacccc catgaacaga | 2580 |
| aatccccctt acacggaggc atcagtgacc aaacaggaaa aaaccgccct taacatggcc | 2640 |
| cgctttatca gaagccagac attaacgctt ctggagaaac tcaacgagct ggacgcggat | 2700 |
| gaacaggcag acatctgtga atcgcttcac gaccacgctg atgagcttta ccgcagctgc | 2760 |
| ctcgcgcgtt tcggtgatga cggtgaaaac ctctgacaca tgcagctccc ggagacggtc | 2820 |
| acagcttgtc tgtaagcgga tgccgggagc agacaagccc gtcagggcgc gtcagcgggt | 2880 |
| gttggcgggt gtcggggcgc agccatgacc cagtcacgta gcgatagcgg agtgtatact | 2940 |
| ggcttaacta tgcggcatca gagcagattg tactgagagt gcaccatata tgcggtgtga | 3000 |
| aataccgcac agatgcgtaa ggagaaaata ccgcatcagg cgctcttccg cttcctcgct | 3060 |
| cactgactcg ctgcgctcgg tcgttcggct gcggcgagcg gtatcagctc actcaaaggc | 3120 |
| ggtaatacgg ttatccacag aatcagggga taacgcagga aagaacatgt gagcaaaagg | 3180 |
| ccagcaaaag gccaggaacc gtaaaaaggc cgcgttgctg gcgtttttcc ataggctccg | 3240 |
| cccccctgac gagcatcaca aaaatcgacg ctcaagtcag aggtggcgaa acccgacagg | 3300 |
| actataaaga taccaggcgt ttccccctgg aagctccctc gtgcgctctc ctgttccgac | 3360 |
| cctgccgctt accggatacc tgtccgcctt tctcccttcg ggaagcgtgg cgctttctca | 3420 |
| tagctcacgc tgtaggtatc tcagttcggt gtaggtcgtt cgctccaagc tgggctgtgt | 3480 |
| gcacgaaccc cccgttcagc ccgaccgctg cgccttatcc ggtaactatc gtcttgagtc | 3540 |
| caacccggta agacacgact tatcgccact ggcagcagcc actggtaaca ggattagcag | 3600 |
| agcgaggtat gtaggcggtg ctacagagtt cttgaagtgg tggcctaact acggctacac | 3660 |
| tagaaggaca gtatttggta tctgcgctct gctgaagcca gttaccttcg gaaaaagagt | 3720 |
| tggtagctct tgatccggca aacaaaccac cgctggtagc ggtggttttt ttgtttgcaa | 3780 |
| gcagcagatt acgcgcagaa aaaaaggatc tcaagaagat cctttgatct tttctacggg | 3840 |
| gtctgacgct cagtggaacg aaaactcacg ttaagggatt ttggtcatga acaataaaac | 3900 |
| tgtctgctta cataaacagt aatacaaggg gtgttatgag ccatattcaa cgggaaacgt | 3960 |
| cttgctctag gccgcgatta aattccaaca tggatgctga tttatatggg tataaatggg | 4020 |
| ctcgcgataa tgtcgggcaa tcaggtgcga caatctatcg attgtatggg aagcccgatg | 4080 |
| cgccagagtt gtttctgaaa catggcaaag gtagcgttgc caatgatgtt acagatgaga | 4140 |
| tggtcagact aaactggctg acggaattta tgcctcttcc gaccatcaag cattttatcc | 4200 |
| gtactcctga tgatgcatgg ttactcacca ctgcgatccc cgggaaaaca gcattccagg | 4260 |
| tattagaaga atatcctgat tcaggtgaaa atattgttga tgcgctggca gtgttcctgc | 4320 |
| gccggttgca ttcgattcct gtttgtaatt gtccttttaa cagcgatcgc gtatttcgtc | 4380 |
| tcgctcaggc gcaatcacga atgaataacg gtttggttga tgcgagtgat tttgatgacg | 4440 |
| agcgtaatgg ctggcctgtt gaacaagtct ggaaagaaat gcataaactt ttgccattct | 4500 |
| caccggattc agtcgtcact catggtgatt tctcacttga taaccttatt tttgacgagg | 4560 |
| ggaaattaat aggttgtatt gatgttggac gagtcggaat cgcagaccga taccaggatc | 4620 |
| ttgccatcct atggaactgc ctcggtgagt tttctccttc attacagaaa cggctttttc | 4680 |
| aaaaatatgg tattgataat cctgatatga ataaattgca gtttcatttg atgctcgatg | 4740 |
| agtttttcta agaattaatt catgagcgga tacatatttg aatgtattta gaaaaataaa | 4800 |
| caaatagggg ttccgcgcac atttccccga aaagtgccac ctgaaattgt aaacgttaat | 4860 |
| attttgttaa aattcgcgtt aaatttttgt taaatcagct cattttttaa ccaataggcc | 4920 |
| gaaatcggca aaatccctta taaatcaaaa gaatagaccg agatagggtt gagtgttgtt | 4980 |
| ccagtttgga acaagagtcc actattaaag aacgtggact ccaacgtcaa agggcgaaaa | 5040 |
| accgtctatc agggcgatgg cccactacgt gaaccatcac cctaatcaag ttttttgggg | 5100 |
| tcgaggtgcc gtaaagcact aaatcggaac cctaaaggga gcccccgatt tagagcttga | 5160 |
| cggggaaagc cggcgaacgt ggcgagaaag gaagggaaga aagcgaaagg agcgggcgct | 5220 |
| agggcgctgg caagtgtagc ggtcacgctg cgcgtaacca ccacacccgc cgcgcttaat | 5280 |
| gcgccgctac agggcgcgtc ccattcgcca | 5310 |

SEQ ID NO: 10

| | |
|---|---|
| tggcgaatgg gacgcgccct gtagcggcgc attaagcgcg gcgggtgtgg tggttacgcg | 60 |
| cagcgtgacc gctacacttg ccagcgccct agcgcccgct cctttcgctt tcttcccttc | 120 |
| ctttctcgcc acgttcgccg gctttccccg tcaagctcta aatcgggggc tccctttagg | 180 |
| gttccgattt agtgctttac ggcacctcga ccccaaaaaa cttgattagg gtgatggttc | 240 |
| acgtagtggg ccatcgccct gatagacggt ttttcgccct ttgacgttgg agtccacgtt | 300 |
| ctttaatagt ggactcttgt tccaaactgg aacaacactc aaccctatct cggtctattc | 360 |
| ttttgattta taagggattt tgccgatttc ggcctattgg ttaaaaaatg agctgattta | 420 |
| acaaaaattt aacgcgaatt ttaacaaaat attaacgttt acaatttcag gtggcacttt | 480 |
| tcggggaaat gtgcgcggaa cccctatttg tttatttttc taaatacatt caaatatgta | 540 |
| tccgctcatg agacaataac cctgataaat gcttcaataa tattgaaaaa ggaagagtat | 600 |
| gagtattcaa catttccgtg tcgcccttat tccctttttt gcggcatttt gccttcctgt | 660 |
| ttttgctcac ccagaaacgc tggtgaaagt aaaagatgct gaagatcagt tgggtgcacg | 720 |
| agtgggttac atcgaactgg atctcaacag cggtaagatc cttgagagtt ttcgccccga | 780 |
| agaacgtttt ccaatgatga gcacttttaa agttctgcta tgtggcgcgg tattatcccg | 840 |
| tattgacgcc gggcaagagc aactcggtcg ccgcatacac tattctcaga atgacttggt | 900 |
| tgagtactca ccagtcacag aaaagcatct tacggatggc atgacagtaa gagaattatg | 960 |
| cagtgctgcc ataaccatga gtgataacac tgcggccaac ttacttctga caacgatcgg | 1020 |
| aggaccgaag gagctaaccg cttttttgca caacatgggg gatcatgtaa ctcgccttga | 1080 |
| tcgttgggaa ccggagctga atgaagccat accaaacgac gagcgtgaca ccacgatgcc | 1140 |
| tgcagcaatg gcaacaacgt tgcgcaaact attaactggc gaactactta ctctagcttc | 1200 |
| ccggcaacaa ttaatagact ggatggaggc ggataaagtt gcaggaccac ttctgcgctc | 1260 |
| ggcccttccg gctggctggt ttattgctga taaatctgga gccggtgagc gtgggtctcg | 1320 |
| cggtatcatt gcagcactgg ggccagatgg taagccctcc cgtatcgtag ttatctacac | 1380 |
| gacggggagt caggcaacta tggatgaacg aaatagacag atcgctgaga taggtgcctc | 1440 |
| actgattaag cattggtaac tgtcagacca agtttactca tatatacttt agattgattt | 1500 |
| aaaacttcat ttttaattta aaaggatcta ggtgaagatc ctttttgata atctcatgac | 1560 |
| caaaatccct taacgtgagt tttcgttcca ctgagcgtca gaccccgtag aaaagatcaa | 1620 |
| aggatcttct tgagatcctt tttttctgcg cgtaatctgc tgcttgcaaa caaaaaaacc | 1680 |
| accgctacca gcggtggttt gtttgccgga tcaagagcta ccaactcttt ttccgaaggt | 1740 |
| aactggcttc agcagagcgc agataccaaa tactgtcctt ctagtgtagc cgtagttagg | 1800 |
| ccaccacttc aagaactctg tagcaccgcc tacatacctc gctctgctaa tcctgttacc | 1860 |
| agtggctgct gccagtggcg ataagtcgtg tcttaccggg ttggactcaa gacgatagtt | 1920 |
| accggataag gcgcagcggt cgggctgaac ggggggttcg tgcacacagc ccagcttgga | 1980 |
| gcgaacgacc tacaccgaac tgagatacct acagcgtgag ctatgagaaa gcgccacgct | 2040 |
| tcccgaaggg agaaaggcgg acaggtatcc ggtaagcggc agggtcggaa caggagagcg | 2100 |
| cacgagggag cttccagggg gaaacgcctg gtatctttat agtcctgtcg ggtttcgcca | 2160 |
| cctctgactt gagcgtcgat ttttgtgatg ctcgtcaggg gggcggagcc tatggaaaaa | 2220 |
| cgccagcaac gcggcctttt tacggttcct ggccttttgc tggccttttg ctcacatgtt | 2280 |
| ctttcctgcg ttatcccctg attctgtgga taaccgtatt accgcctttg agtgagctga | 2340 |
| taccgctcgc cgcagccgaa cgaccgagcg cagcgagtca gtgagcgagg aagcggaaga | 2400 |
| gcgcctgatg cggtattttc tccttacgca tctgtgcggt atttcacacc gcatatatgg | 2460 |
| tgcactctca gtacaatctg ctctgatgcc gcatagttaa gccagtatac actccgctat | 2520 |
| cgctacgtga ctgggtcatg gctgcgcccc gacacccgcc aacacccgct gacgcgccct | 2580 |
| gacgggcttg tctgctcccg gcatccgctt acagacaagc tgtgaccgtc tccgggagct | 2640 |
| gcatgtgtca gaggttttca ccgtcatcac cgaaacgcgc gaggcagctg cggtaaagct | 2700 |
| catcagcgtg gtcgtgaagc gattcacaga tgtctgcctg ttcatccgcg tccagctcgt | 2760 |
| tgagtttctc cagaagcgtt aatgtctggc ttctgataaa gcgggccatg ttaagggcgg | 2820 |
| ttttttcctg tttggtcact gatgcctccg tgtaaggggg atttctgttc atgggggtaa | 2880 |
| tgataccgat gaaacgagag aggatgctca cgatacgggt tactgatgat gaacatgccc | 2940 |
| ggttactgga acgttgtgag ggtaaacaac tggcggtatg gatgcggcgg gaccagagaa | 3000 |
| aaatcactca gggtcaatgc cagcgcttcg ttaatacaga tgtaggtgtt ccacagggta | 3060 |
| gccagcagca tcctgcgatg cagatccgga acataatggt gcagggcgct gacttccgcg | 3120 |
| tttccagact ttacgaaaca cggaaaccga agaccattca tgttgttgct caggtcgcag | 3180 |
| acgttttgca gcagcagtcg cttcacgttc gctcgcgtat cggtgattca ttctgctaac | 3240 |
| cagtaaggca accccgccag cctagccggg tcctcaacga caggagcacg atcatgcgca | 3300 |
| cccgtggggc cgccatgccg gcgataatgg cctgcttctc gccgaaacgt ttggtggcgg | 3360 |
| gaccagtgac gaaggcttga gcgagggcgt gcaagattcc gaataccgca agcgacaggc | 3420 |
| cgatcatcgt cgcgctccag cgaaagcggt cctcgccgaa aatgacccag agcgctgccg | 3480 |
| gcacctgtcc tacgagttgc atgataaaga agacagtcat aagtgcggcg acgatagtca | 3540 |
| tgccccgcgc ccaccggaag gagctgactg ggttgaaggc tctcaagggc atcggtcgag | 3600 |
| atcccggtgc ctaatgagtg agctaactta cattaattgc gttgcgctca ctgcccgctt | 3660 |
| tccagtcggg aaacctgtcg tgccagctgc attaatgaat cggccaacgc gcggggagag | 3720 |
| gcggtttgcg tattgggcgc cagggtggtt tttcttttca ccagtgagac gggcaacagc | 3780 |
| tgattgccct tcaccgcctg gccctgagag agttgcagca agcggtccac gctggtttgc | 3840 |
| cccagcaggc gaaaatcctg tttgatggtg gttaacggcg ggatataaca tgagctgtct | 3900 |
| tcggtatcgt cgtatcccac taccgagata tccgcaccaa cgcgcagccc ggactcggta | 3960 |
| atggcgcgca ttgcgcccag cgccatctga tcgttggcaa ccagcatcgc agtgggaacg | 4020 |
| atgccctcat tcagcatttg catggtttgt tgaaaaccgg acatggcact ccagtcgcct | 4080 |
| tcccgttccg ctatcggctg aatttgattg cgagtgagat atttatgcca gccagccaga | 4140 |
| cgcagacgcg ccgagacaga acttaatggg cccgctaaca gcgcgatttg ctggtgaccc | 4200 |
| aatgcgacca gatgctccac gcccagtcgc gtaccgtctt catgggagaa aataatactg | 4260 |
| ttgatgggtg tctggtcaga gacatcaaga aataacgccg gaacattagt gcaggcagct | 4320 |
| tccacagcaa tggcatcctg gtcatccagc ggatagttaa tgatcagccc actgacgcgt | 4380 |
| tgcgcgagaa gattgtgcac cgccgcttta caggcttcga cgccgcttcg ttctaccatc | 4440 |
| gacaccacca cgctggcacc cagttgatcg gcgcgagatt taatcgccgc gacaatttgc | 4500 |
| gacggcgcgt gcagggccag actggaggtg gcaacgccaa tcagcaacga ctgtttgccc | 4560 |
| gccagttgtt gtgccacgcg gttgggaatg taattcagct ccgccatcgc cgcttccact | 4620 |
| ttttcccgcg ttttcgcaga aacgtggctg gcctggttca ccacgcggga aacggtctga | 4680 |
| taagagacac cggcatactc tgcgacatcg tataacgtta ctggtttcac attcaccacc | 4740 |
| ctgaattgac tctcttccgg gcgctatcat gccataccgc gaaaggtttt gcgccattcg | 4800 |
| atggtgtccg ggatctcgac gctctccctt atgcgactcc tgcattagga agcagcccag | 4860 |
| tagtaggttg aggccgttga gcaccgccgc cgcaaggaat ggtgcatgca aggagatggc | 4920 |
| gcccaacagt cccccggcca cggggcctgc caccataccc acgccgaaac aagcgctcat | 4980 |
| gagcccgaag tggcgagccc gatcttcccc atcggtgatg tcggcgatat aggcgccagc | 5040 |
| aaccgcacct gtggcgccgg tgatgccggc cacgatgcgt ccggcgtaga ggatcgagat | 5100 |
| ctcgatcccg cgaaattaat acgactcact ataggggaat tgtgagcgga taacaattcc | 5160 |
| cctctagaaa taattttgtt taactttaag aaggagatat acatatggct agcatgactg | 5220 |
| gtggacagca aatgggtcgc ggatccgaat tcgagctccg tcgacaagct tgcggccgca | 5280 |
| ctcgagcacc accaccacca ccactgagat ccggctgcta acaaagcccg aaaggaagct | 5340 |
| gagttggctg ctgccaccgc tgagcaataa ctagcataac cccttggggc ctctaaacgg | 5400 |
| gtcttgaggg gttttttgct gaaaggagga actatatccg gat | 5443 |

SEQ ID NO: 11

| | |
|---|---|
| atccggatat agttcctcct ttcagcaaaa aacccctcaa gacccgttta gaggccccaa | 60 |
| ggggttatgc tagttattgc tcagcggtgg cagcagccaa ctcagcttcc tttcgggctt | 120 |
| tgttagcagc cggatctcag tggtggtggt ggtggtgctc gagggatcaa tactaggagg | 180 |
| agtagcatat aattacgtta cacaatttta taacccaata tattcaatag accttatgct | 240 |
| tatcctatcc tctattctaa gattctcggt atctccccta ttcttgacca taaaagatac | 300 |
| tcgctcaaag cttaaataat attaatcata aataaagtca tgtactcatt tccaaatagc | 360 |
| tttaggtttg gttggtccca ggccggattt caatcagaaa tgggaacacc agggtcagaa | 420 |
| gatccaaata ctgactggta taaatgggtt catgatccag aaaacatggc agcgggatta | 480 |
| gtaagtggag atctaccaga aaatgggcca ggctactggg gaaactataa gacatttcac | 540 |
| gataatgcac aaaaaatggg attaaaaata gctagactaa atgtggaatg gtctaggata | 600 |
| tttcctaatc cattaccaag gccacaaaac tttgatgaat caaaacaaga tgtgacagag | 660 |
| gttgagataa acgaaaacga gttaaagaga cttgacgagt acgctaataa agacgcatta | 720 |
| aaccattaca gggaaatatt caaggatctt aaaagtagag gactttactt tatactaaac | 780 |
| atgtatcatt ggccattacc tctatggtta cacgacccaa taagagtaag aagaggagat | 840 |
| tttactggac caagtggttg gctaagtact agaacagttt acgaattcgc tagattctca | 900 |
| gcttatatag cttggaaatt cgatgatcta gtggatgagt actcaacaat gaatgaacct | 960 |
| aacgttgttg gaggtttagg atacgttggt gttaagtccg gttttccccc aggataccta | 1020 |
| agctttgaac tttcccgtag ggcaatgtat aacatcattc aagctcacgc aagagcgtat | 1080 |
| gatgggataa agagtgtttc taaaaaacca gttggaatta tttacgctaa tagctcattc | 1140 |
| cagccgttaa cggataaaga tatggaagcg gtagagatgg ctgaaaatga taatagatgg | 1200 |
| tggttctttg atgctataat aagaggtgag atcaccagag gaaacgagaa gattgtaaga | 1260 |
| gatgacctaa agggtagatt ggattggatt ggagttaatt attacactag gactgttgtg | 1320 |
| aagaggactg aaaagggata cgttagctta ggaggttacg gtcacggatg tgagaggaat | 1380 |
| tctgtaagtt tagcgggatt accaaccagc gacttcggct gggagttctt cccagaaggt | 1440 |
| ttatatgacg ttttgacgaa atactggaat agatatcatc tctatatgta cgttactgaa | 1500 |
| aatggtattg cggatgatgc cgattatcaa aggccctatt atttagtatc tcacgtttat | 1560 |
| caagttcata gagcaataaa tagtggtgca gatgttagag ggtatttaca ttggtctcta | 1620 |
| gctgataatt acgaatgggc ttcaggattc tctatgaggt ttggtctgtt aaaggtcgat | 1680 |
| tacaacacta agagactata ctggagaccc tcagcactag tatataggga aatcgccaca | 1740 |
| aatggcgcaa taactgatga aatagagcac ttaaatagcg tacctccagt aaagccatta | 1800 |
| aggcactaaa ctttctcaag tctcactata ccaaatgagt tttcttttaa tcttattcta | 1860 |
| atctcatttt cattagattg caatactttc ataccttcta tattatttat tttgtacctt | 1920 |
| ttgggatcca tatgtatatc tccttcttaa agttaaacaa aattatttct agaggggaat | 1980 |
| tgttatccgc tcacaattcc cctatagtga gtcgtattaa tttcgcggga tcgagatctc | 2040 |
| gatcctctac gccggacgca tcgtggccgg catcaccggc gccacaggtg cggttgctgg | 2100 |
| cgcctatatc gccgacatca ccgatgggga agatcgggct cgccacttcg ggctcatgag | 2160 |
| cgcttgtttc ggcgtgggta tggtggcagg ccccgtggcc gggggactgt tgggcgccat | 2220 |
| ctccttgcat gcaccattcc ttgcggcggc ggtgctcaac ggcctcaacc tactactggg | 2280 |
| ctgcttccta atgcaggagt cgcataaggg agagcgtcga gatcccggac accatcgaat | 2340 |
| ggcgcaaaac ctttcgcggt atggcatgat agcgcccgga agagagtcaa ttcagggtgg | 2400 |
| tgaatgtgaa accagtaacg ttatacgatg tcgcagagta tgccggtgtc tcttatcaga | 2460 |
| ccgtttcccg cgtggtgaac caggccagcc acgtttctgc gaaaacgcgg gaaaaagtgg | 2520 |
| aagcggcgat ggcggagctg aattacattc ccaaccgcgt ggcacaacaa ctggcgggca | 2580 |
| aacagtcgtt gctgattggc gttgccacct ccagtctggc cctgcacgcg ccgtcgcaaa | 2640 |
| ttgtcgcggc gattaaatct cgcgccgatc aactgggtgc cagcgtggtg gtgtcgatgg | 2700 |
| tagaacgaag cggcgtcgaa gcctgtaaag cggcggtgca caatcttctc gcgcaacgcg | 2760 |
| tcagtgggct gatcattaac tatccgctgg atgaccagga tgccattgct gtggaagctg | 2820 |
| cctgcactaa tgttccggcg ttatttcttg atgtctctga ccagacaccc atcaacagta | 2880 |
| ttattttctc ccatgaagac ggtacgcgac tgggcgtgga gcatctggtc gcattgggtc | 2940 |
| accagcaaat cgcgctgtta gcgggcccat taagttctgt ctcggcgcgt ctgcgtctgg | 3000 |
| ctggctggca taaatatctc actcgcaatc aaattcagcc gatagcggaa cgggaaggcg | 3060 |
| actggagtgc catgtccggt tttcaacaaa ccatgcaaat gctgaatgag ggcatcgttc | 3120 |
| ccactgcgat gctggttgcc aacgatcaga tggcgctggg cgcaatgcgc gccattaccg | 3180 |
| agtccgggct gcgcgttggt gcggatatct cggtagtggg atacgacgat accgaagaca | 3240 |
| gctcatgtta tatcccgccg ttaaccacca tcaaacagga ttttcgcctg ctggggcaaa | 3300 |
| ccagcgtgga ccgcttgctg caactctctc agggccaggc ggtgaagggc aatcagctgt | 3360 |
| tgcccgtctc actggtgaaa agaaaaacca ccctggcgcc caatacgcaa accgcctctc | 3420 |
| cccgcgcgtt ggccgattca ttaatgcagc tggcacgaca ggtttcccga ctggaaagcg | 3480 |
| ggcagtgagc gcaacgcaat taatgtaagt tagctcactc attaggcacc gggatctcga | 3540 |
| ccgatgccct tgagagcctt caacccagtc agctccttcc ggtgggcgcg gggcatgact | 3600 |
| atcgtcgccg cacttatgac tgtcttcttt atcatgcaac tcgtaggaca ggtgccggca | 3660 |
| gcgctctggg tcattttcgg cgaggaccgc tttcgctgga gcgcgacgat gatcggcctg | 3720 |
| tcgcttgcgg tattcggaat cttgcacgcc ctcgctcaag ccttcgtcac tggtcccgcc | 3780 |
| accaaacgtt tcggcgagaa gcaggccatt atcgccggca tggcggcccc acgggtgcgc | 3840 |
| atgatcgtgc tcctgtcgtt gaggacccgg ctaggctggc ggggttgcct tactggttag | 3900 |
| cagaatgaat caccgatacg cgagcgaacg tgaagcgact gctgctgcaa aacgtctgcg | 3960 |
| acctgagcaa caacatgaat ggtcttcggt ttccgtgttt cgtaaagtct ggaaacgcgg | 4020 |
| aagtcagcgc cctgcaccat tatgttccgg atctgcatcg caggatgctg ctggctaccc | 4080 |
| tgtggaacac ctacatctgt attaacgaag cgctggcatt gaccctgagt gatttttctc | 4140 |
| tggtcccgcc gcatccatac cgccagttgt ttaccctcac aacgttccag taaccgggca | 4200 |
| tgttcatcat cagtaacccg tatcgtgagc atcctctctc gtttcatcgg tatcattacc | 4260 |
| cccatgaaca gaaatccccc ttacacggag gcatcagtga ccaaacagga aaaaaccgcc | 4320 |
| cttaacatgg cccgctttat cagaagccag acattaacgc ttctggagaa actcaacgag | 4380 |
| ctggacgcgg atgaacaggc agacatctgt gaatcgcttc acgaccacgc tgatgagctt | 4440 |
| taccgcagct gcctcgcgcg tttcggtgat gacggtgaaa acctctgaca catgcagctc | 4500 |
| ccggagacgg tcacagcttg tctgtaagcg gatgccggga gcagacaagc ccgtcagggc | 4560 |
| gcgtcagcgg gtgttggcgg gtgtcggggc gcagccatga cccagtcacg tagcgatagc | 4620 |
| ggagtgtata ctggcttaac tatgcggcat cagagcagat tgtactgaga gtgcaccata | 4680 |
| tatgcggtgt gaaataccgc acagatgcgt aaggagaaaa taccgcatca ggcgctcttc | 4740 |
| cgcttcctcg ctcactgact cgctgcgctc ggtcgttcgg ctgcggcgag cggtatcagc | 4800 |
| tcactcaaag gcggtaatac ggttatccac agaatcaggg gataacgcag gaaagaacat | 4860 |
| gtgagcaaaa ggccagcaaa aggccaggaa ccgtaaaaag gccgcgttgc tggcgttttt | 4920 |
| ccataggctc cgcccccctg acgagcatca caaaaatcga cgctcaagtc agaggtggcg | 4980 |
| aaacccgaca ggactataaa gataccaggc gtttccccct ggaagctccc tcgtgcgctc | 5040 |
| tcctgttccg accctgccgc ttaccggata cctgtccgcc tttctccctt cgggaagcgt | 5100 |
| ggcgctttct catagctcac gctgtaggta tctcagttcg gtgtaggtcg ttcgctccaa | 5160 |
| gctgggctgt gtgcacgaac cccccgttca gcccgaccgc tgcgccttat ccggtaacta | 5220 |
| tcgtcttgag tccaacccgg taagacacga cttatcgcca ctggcagcag ccactggtaa | 5280 |
| caggattagc agagcgaggt atgtaggcgg tgctacagag ttcttgaagt ggtggcctaa | 5340 |
| ctacggctac actagaagga cagtatttgg tatctgcgct ctgctgaagc cagttacctt | 5400 |
| cggaaaaaga gttggtagct cttgatccgg caaacaaacc accgctggta gcggtggttt | 5460 |
| ttttgtttgc aagcagcaga ttacgcgcag aaaaaaagga tctcaagaag atcctttgat | 5520 |
| cttttctacg gggtctgacg ctcagtggaa cgaaaactca cgttaaggga ttttggtcat | 5580 |
| gaacaataaa actgtctgct tacataaaca gtaatacaag gggtgttatg agccatattc | 5640 |
| aacgggaaac gtcttgctct aggccgcgat taaattccaa catggatgct gatttatatg | 5700 |
| ggtataaatg ggctcgcgat aatgtcgggc aatcaggtgc gacaatctat cgattgtatg | 5760 |
| ggaagcccga tgcgccagag ttgtttctga aacatggcaa aggtagcgtt gccaatgatg | 5820 |
| ttacagatga gatggtcaga ctaaactggc tgacggaatt tatgcctctt ccgaccatca | 5880 |
| agcattttat ccgtactcct gatgatgcat ggttactcac cactgcgatc cccgggaaaa | 5940 |
| cagcattcca ggtattagaa gaatatcctg attcaggtga aaatattgtt gatgcgctgg | 6000 |
| cagtgttcct gcgccggttg cattcgattc ctgtttgtaa ttgtcctttt aacagcgatc | 6060 |
| gcgtatttcg tctcgctcag gcgcaatcac gaatgaataa cggtttggtt gatgcgagtg | 6120 |
| attttgatga cgagcgtaat ggctggcctg ttgaacaagt ctggaaagaa atgcataaac | 6180 |
| ttttgccatt ctcaccggat tcagtcgtca ctcatggtga tttctcactt gataacctta | 6240 |
| tttttgacga ggggaaatta ataggttgta ttgatgttgg acgagtcgga atcgcagacc | 6300 |
| gataccagga tcttgccatc ctatggaact gcctcggtga gttttctcct tcattacaga | 6360 |
| aacggctttt tcaaaaatat ggtattgata atcctgatat gaataaattg cagtttcatt | 6420 |
| tgatgctcga tgagtttttc taagaattaa ttcatgagcg gatacatatt tgaatgtatt | 6480 |
| tagaaaaata aacaaatagg ggttccgcgc acatttcccc gaaaagtgcc acctgaaatt | 6540 |
| gtaaacgtta atattttgtt aaaattcgcg ttaaattttt gttaaatcag ctcatttttt | 6600 |
| aaccaatagg ccgaaatcgg caaaatccct tataaatcaa aagaatagac cgagataggg | 6660 |
| ttgagtgttg ttccagtttg gaacaagagt ccactattaa agaacgtgga ctccaacgtc | 6720 |
| aaagggcgaa aaaccgtcta tcagggcgat ggcccactac gtgaaccatc accctaatca | 6780 |
| agttttttgg ggtcgaggtg ccgtaaagca ctaaatcgga accctaaagg gagcccccga | 6840 |
| tttagagctt gacggggaaa gccggcgaac gtggcgagaa aggaagggaa gaaagcgaaa | 6900 |
| ggagcgggcg ctagggcgct ggcaagtgta gcggtcacgc tgcgcgtaac caccacaccc | 6960 |
| gccgcgctta atgcgccgct acagggcgcg tcccattcgc ca | 7002 |

SEQ ID NO: 12

| | |
|---|---|
| ggcgaatggg acgcgccctg tagcggcgca ttaagcgcgg cgggtgtggt ggttacgcgc | 60 |
| agcgtgaccg ctacacttgc cagcgcccta gcgcccgctc ctttcgcttt cttcccttcc | 120 |
| tttctcgcca cgttcgccgg ctttccccgt caagctctaa atcgggggct ccctttaggg | 180 |
| ttccgattta gtgctttacg gcacctcgac cccaaaaaac ttgattaggg tgatggttca | 240 |
| cgtagtgggc catcgccctg atagacggtt tttcgccctt tgacgttgga gtccacgttc | 300 |
| tttaatagtg gactcttgtt ccaaactgga acaacactca accctatctc ggtctattct | 360 |
| tttgatttat aagggatttt gccgatttcg gcctattggt taaaaaatga gctgatttaa | 420 |
| caaaaattta acgcgaattt taacaaaata ttaacgttta caatttcagg tggcactttt | 480 |
| cggggaaatg tgcgcggaac ccctatttgt ttatttttct aaatacattc aaatatgtat | 540 |
| ccgctcatga gacaataacc ctgataaatg cttcaataat attgaaaaag gaagagtatg | 600 |
| agtattcaac atttccgtgt cgcccttatt cccttttttg cggcattttg ccttcctgtt | 660 |
| tttgctcacc cagaaacgct ggtgaaagta aaagatgctg aagatcagtt gggtgcacga | 720 |
| gtgggttaca tcgaactgga tctcaacagc ggtaagatcc ttgagagttt tcgccccgaa | 780 |
| gaacgttttc caatgatgag cacttttaaa gttctgctat gtggcgcggt attatcccgt | 840 |
| attgacgccg ggcaagagca actcggtcgc cgcatacact attctcagaa tgacttggtt | 900 |
| gagtactcac cagtcacaga aaagcatctt acggatggca tgacagtaag agaattatgc | 960 |
| agtgctgcca taaccatgag tgataacact gcggccaact tacttctgac aacgatcgga | 1020 |
| ggaccgaagg agctaaccgc ttttttgcac aacatggggg atcatgtaac tcgccttgat | 1080 |
| cgttgggaac cggagctgaa tgaagccata ccaaacgacg agcgtgacac cacgatgcct | 1140 |
| gcagcaatgg caacaacgtt gcgcaaacta ttaactggcg aactacttac tctagcttcc | 1200 |
| cggcaacaat taatagactg gatggaggcg gataaagttg caggaccact tctgcgctcg | 1260 |
| gcccttccgg ctggctggtt tattgctgat aaatctggag ccggtgagcg tgggtctcgc | 1320 |
| ggtatcattg cagcactggg gccagatggt aagccctccc gtatcgtagt tatctacacg | 1380 |
| acggggagtc aggcaactat ggatgaacga aatagacaga tcgctgagat aggtgcctca | 1440 |
| ctgattaagc attggtaact gtcagaccaa gtttactcat atatacttta gattgattta | 1500 |
| aaacttcatt tttaatttaa aaggatctag gtgaagatcc tttttgataa tctcatgacc | 1560 |
| aaaatccctt aacgtgagtt ttcgttccac tgagcgtcag accccgtaga aaagatcaaa | 1620 |
| ggatcttctt gagatccttt ttttctgcgc gtaatctgct gcttgcaaac aaaaaaacca | 1680 |
| ccgctaccag cggtggtttg tttgccggat caagagctac caactctttt tccgaaggta | 1740 |
| actggcttca gcagagcgca gataccaaat actgtccttc tagtgtagcc gtagttaggc | 1800 |
| caccacttca agaactctgt agcaccgcct acatacctcg ctctgctaat cctgttacca | 1860 |
| gtggctgctg ccagtggcga taagtcgtgt cttaccgggt tggactcaag acgatagtta | 1920 |
| ccggataagg cgcagcggtc gggctgaacg gggggttcgt gcacacagcc cagcttggag | 1980 |
| cgaacgacct acaccgaact gagataccta cagcgtgagc tatgagaaag cgccacgctt | 2040 |
| cccgaaggga gaaaggcgga caggtatccg gtaagcggca gggtcggaac aggagagcgc | 2100 |
| acgagggagc ttccaggggg aaacgcctgg tatctttata gtcctgtcgg gtttcgccac | 2160 |
| ctctgacttg agcgtcgatt tttgtgatgc tcgtcagggg ggcggagcct atggaaaaac | 2220 |
| gccagcaacg cggccttttt acggttcctg gccttttgct ggccttttgc tcacatgttc | 2280 |
| tttcctgcgt tatcccctga ttctgtggat aaccgtatta ccgcctttga gtgagctgat | 2340 |
| accgctcgcc gcagccgaac gaccgagcgc agcgagtcag tgagcgagga agcggaagag | 2400 |
| cgcctgatgc ggtattttct ccttacgcat ctgtgcggta tttcacaccg catatatggt | 2460 |
| gcactctcag tacaatctgc tctgatgccg catagttaag ccagtataca ctccgctatc | 2520 |
| gctacgtgac tgggtcatgg ctgcgccccg acacccgcca acacccgctg acgcgccctg | 2580 |
| acgggcttgt ctgctcccgg catccgctta cagacaagct gtgaccgtct ccgggagctg | 2640 |
| catgtgtcag aggttttcac cgtcatcacc gaaacgcgcg aggcagctgc ggtaaagctc | 2700 |
| atcagcgtgg tcgtgaagcg attcacagat gtctgcctgt tcatccgcgt ccagctcgtt | 2760 |
| gagtttctcc agaagcgtta atgtctggct tctgataaag cgggccatgt taagggcggt | 2820 |
| tttttcctgt ttggtcactg atgcctccgt gtaaggggga tttctgttca tgggggtaat | 2880 |
| gataccgatg aaacgagaga ggatgctcac gatacgggtt actgatgatg aacatgcccg | 2940 |
| gttactggaa cgttgtgagg gtaaacaact ggcggtatgg atgcggcggg accagagaaa | 3000 |
| aatcactcag ggtcaatgcc agcgcttcgt taatacagat gtaggtgttc cacagggtag | 3060 |
| ccagcagcat cctgcgatgc agatccggaa cataatggtg cagggcgctg acttccgcgt | 3120 |
| ttccagactt tacgaaacac ggaaaccgaa gaccattcat gttgttgctc aggtcgcaga | 3180 |
| cgttttgcag cagcagtcgc ttcacgttcg ctcgcgtatc ggtgattcat tctgctaacc | 3240 |
| agtaaggcaa ccccgccagc ctagccgggt cctcaacgac aggagcacga tcatgcgcac | 3300 |
| ccgtggggcc gccatgccgg cgataatggc ctgcttctcg ccgaaacgtt tggtggcggg | 3360 |
| accagtgacg aaggcttgag cgagggcgtg caagattccg aataccgcaa gcgacaggcc | 3420 |
| gatcatcgtc gcgctccagc gaaagcggtc ctcgccgaaa atgacccaga gcgctgccgg | 3480 |
| cacctgtcct acgagttgca tgataaagaa gacagtcata agtgcggcga cgatagtcat | 3540 |
| gccccgcgcc caccggaagg agctgactgg gttgaaggct ctcaagggca tcggtcgaga | 3600 |
| tcccggtgcc taatgagtga gctaacttac attaattgcg ttgcgctcac tgcccgcttt | 3660 |
| ccagtcggga aacctgtcgt gccagctgca ttaatgaatc ggccaacgcg cggggagagg | 3720 |
| cggtttgcgt attgggcgcc agggtggttt ttcttttcac cagtgagacg ggcaacagct | 3780 |
| gattgccctt caccgcctgg ccctgagaga gttgcagcaa gcggtccacg ctggtttgcc | 3840 |
| ccagcaggcg aaaatcctgt ttgatggtgg ttaacggcgg gatataacat gagctgtctt | 3900 |
| cggtatcgtc gtatcccact accgagatat ccgcaccaac gcgcagcccg gactcggtaa | 3960 |
| tggcgcgcat tgcgcccagc gccatctgat cgttggcaac cagcatcgca gtgggaacga | 4020 |
| tgccctcatt cagcatttgc atggtttgtt gaaaaccgga catggcactc cagtcgcctt | 4080 |
| cccgttccgc tatcggctga atttgattgc gagtgagata tttatgccag ccagccagac | 4140 |
| gcagacgcgc cgagacagaa cttaatgggc ccgctaacag cgcgatttgc tggtgaccca | 4200 |
| atgcgaccag atgctccacg cccagtcgcg taccgtcttc atgggagaaa ataatactgt | 4260 |
| tgatgggtgt ctggtcagag acatcaagaa ataacgccgg aacattagtg caggcagctt | 4320 |
| ccacagcaat ggcatcctgg tcatccagcg gatagttaat gatcagccca ctgacgcgtt | 4380 |
| gcgcgagaag attgtgcacc gccgctttac aggcttcgac gccgcttcgt tctaccatcg | 4440 |
| acaccaccac gctggcaccc agttgatcgg cgcgagattt aatcgccgcg acaatttgcg | 4500 |
| acggcgcgtg cagggccaga ctggaggtgg caacgccaat cagcaacgac tgtttgcccg | 4560 |
| ccagttgttg tgccacgcgg ttgggaatgt aattcagctc cgccatcgcc gcttccactt | 4620 |
| tttcccgcgt tttcgcagaa acgtggctgg cctggttcac cacgcgggaa acggtctgat | 4680 |
| aagagacacc ggcatactct gcgacatcgt ataacgttac tggtttcaca ttcaccaccc | 4740 |
| tgaattgact ctcttccggg cgctatcatg ccataccgcg aaaggttttg cgccattcga | 4800 |
| tggtgtccgg gatctcgacg ctctccctta tgcgactcct gcattaggaa gcagcccagt | 4860 |
| agtaggttga ggccgttgag caccgccgcc gcaaggaatg gtgcatgcaa ggagatggcg | 4920 |
| cccaacagtc ccccggccac ggggcctgcc accataccca cgccgaaaca agcgctcatg | 4980 |
| agcccgaagt ggcgagcccg atcttcccca tcggtgatgt cggcgatata ggcgccagca | 5040 |
| accgcacctg tggcgccggt gatgccggcc acgatgcgtc cggcgtagag gatcgagatc | 5100 |
| tcgatcccgc gaaattaata cgactcacta taggggaatt gtgagcggat aacaattccc | 5160 |
| ctctagaaat aattttgttt aactttaaga aggagatata catatgatgt cctacaggat | 5220 |
| agtggttgat ccaaaaaaag ttgtcaagcc gattagtaga cacatctacg gtcatttcac | 5280 |
| ggaacatctg ggaaggtgta tctacggcgg aatttatgaa gaaggttctc cgctctccga | 5340 |
| tgaaaggggt ttcagaaagg acgttctgga ggctgtaaag aggataaaag ttccgaactt | 5400 |
| gagatggccc ggtggaaact ttgtgtcgaa ctaccactgg gaagacggaa taggtcccaa | 5460 |
| agatcagagg cctgtcaggt tcgatctcgc ctggcaacag gaagagacga atagatttgg | 5520 |
| aacggacgaa ttcattgagt actgtcgtga gataggagca gaaccttaca tcagtataaa | 5580 |
| catgggaact ggaacactcg acgaagctct ccactggctt gaatactgca atggaaaggg | 5640 |
| taatacctac tacgctcaac tcagaagaaa gtacggtcat ccagaacctt acaacgtaaa | 5700 |
| gttctgggga ataggcaacg agatgtacgg ggaatggcag gtaggccaca tgacggcgga | 5760 |
| cgaatacgca agagccgcca aagaatacac gaaatggatg aaggttttcg atcctacaat | 5820 |
| taaagcgatc gccgtgggct gtgacgaccc tatatggaat ctcagggttc ttcaagaagc | 5880 |
| aggtgatgtg attgacttca tatcctacca tttctacaca gggtccgagg attactacga | 5940 |
| aacagtttcc acggtttacc ttctcaaaga aagactcatc ggagtgaaaa agctcattga | 6000 |
| tatggtggat actgctagaa agagaggtgt caaaatcgcc cttgatgaat ggaacgtatg | 6060 |
| gtacagagtg tccgataaca agctcgaaga accttacgat ctcaaagatg gtatctttgc | 6120 |
| atgtggagtg cttgtacttc ttcaaaagat gagcgacata gtcccacttg ccaatctcgc | 6180 |
| acagcttgta aacgcccttg gagctataca caccgagaaa gacggtctca ttctcacacc | 6240 |
| cgtttacaag gcttttgaac tcatcgtgaa tcattccgga gaaaagcttg tcaagaccca | 6300 |
| tgttgaatcg gagacttaca acatagaagg agtcatgttc atcaacaaaa tgcctttctc | 6360 |
| tgtcgagaac gcaccgttcc ttgatgccgc cgcttccatc tcagaagatg gcaagaaact | 6420 |
| tttcatcgct gttgtaaact acaggaaaga agacgctttg aaggttccaa tcagagtgga | 6480 |
| aggtctggga cagaaaaaag ccaccgttta tacactcaca ggtccggacg tgaacgcgag | 6540 |
| aaacaccatg gaaaatccga acgtcgttga tattacctcc gaaaccatca ccgttgacac | 6600 |
| cgaatttgaa cacacgttta aaccattctc ttgcagtgtg attgaggtag aattggagct | 6660 |
| cgagcaccac caccaccacc actgagatcc ggctgctaac aaagcccgaa aggaagctga | 6720 |
| gttggctgct gccaccgctg agcaataact agcataaccc cttggggcct ctaaacgggt | 6780 |
| cttgaggggt tttttgctga aaggaggaac tatatccgga t | 6821 |

SEQ ID NO: 13

| | |
|---|---|
| atggcagcta aagacgtaaa attcggtaac gacgctcgtg tgaaaatgct gcgcggcgta | 60 |
| aacgtactgg cagatgcagt gaaagttacc ctcggtccga aaggccgtaa cgtagttctg | 120 |
| gataaatctt tcggtgcacc gaccatcacc aaagatggtg tttccgttgc tcgtgaaatc | 180 |
| gaactggaag acaagttcga aaatatgggt gcgcagatgg tgaaagaagt tgcctccaaa | 240 |
| gcgaacgacg ctgcaggcga cggtaccacc actgcaaccg tactggctca ggctatcatc | 300 |
| actgaaggtc tgaaagctgt tgctgcgggc atgaacccga tggacctgaa acgtggtatc | 360 |
| gacaaagcgg ttaccgctgc agttgaagaa ctgaaagcgc tgtccgtacc gtgctctgat | 420 |
| tctaaagcga ttgctcaggt tggtaccatc tccgctaact ccgacgaaac cgtaggtaaa | 480 |
| ctgatcgcag aagcgatgga caaagtcggt aaagaaggcg ttatcaccgt tgaagacggt | 540 |
| accggtctgc aggacgaact ggacgtggtt gaaggtatgc agttcgaccg tggctacctg | 600 |
| tctccttact tcatcaacaa gccggaaact ggcgcagtag aactggaaag cccgttcatc | 660 |
| ctgctggctg acaagaaaat ctccaacatc cgcgaaatgc tgccggttct ggaagctgtt | 720 |
| gcaaaagcag gtaaaccgct gctgatcatc gctgaagatg tagaaggcga agcgctggca | 780 |
| actctggttg ttaacaccat gcgtggcatc gtgaaagtcg ctgcggttaa agcaccgggc | 840 |
| ttcggcgatc gtcgtaaagc tatgctgcag gatatcgcaa ccctgactgg cggtaccgtg | 900 |
| atctctgaag agatcggtat ggagctggaa aaagcaaccc tggaagacct gggtcaggct | 960 |
| aaacgtgttg tgatcaacaa agacaccacc actatcatcg atggcgtggg tgaagaagct | 1020 |
| gcaatccagg gccgtgttgc tcagatccgt cagcagattg aagaagcaac ttctgactac | 1080 |
| gaccgtgaaa aactgcagga acgcgtagcg aaactggcag gcggcgttgc agttatcaaa | 1140 |
| gtaggtgctg ctaccgaagt tgaaatgaaa gagaaaaaag cacgcgttga agatgccctg | 1200 |
| cacgcgaccc gtgcagcggt agaagagggc gtggttgctg gtggtggtgt tgcgctgatc | 1260 |
| cgcgtagcgt ctaaactggc tgacctgcgt ggtcagaacg aagaccagaa cgtgggtatc | 1320 |
| aaagttgcac tgcgtgcaat ggaagctccg ctgcgtcaga tcgtattgaa ctgcggcgaa | 1380 |
| gaaccgtctg ttgttgctaa caccgttaaa ggcggcgacg gcaactacgg ttacaacgca | 1440 |
| gcaaccgaag aatacggcaa catgatcgac atgggtatcc tggatccaac caaagtaact | 1500 |
| cgttctgctc tgcagtacgc agcttctgtg gctggcctga tgatcaccac cgagtgcatg | 1560 |
| gttaccgacc tgccgaaaaa cgatgcagct gacttaggcg ctgctggcgg tatgggcggc | 1620 |
| atgggtggca tgggcggcat gatgtaa | 1647 |

SEQ ID NO: 14

| | |
|---|---|
| atgaatattc gtccattgca tgatcgcgtg atcgtcaagc gtaaagaagt tgaaactaaa | 60 |
| tctgctggcg gcatcgttct gaccggctct gcagcggcta aatccacccg tggcgaagtg | 120 |
| ctggctgtcg gcaatggccg tatccttgaa aatggcgaag tgaagccgct ggatgtgaaa | 180 |
| gttggcgaca tcgttatttt caacgatggc tacggtgtga aatctgagaa gatcgacaat | 240 |
| gaagaagtgt tgatcatgtc cgaaagcgac attctggcaa ttgttgaagc gtaa | 29 |

1. A composition for production of ginsenoside compound K comprising a high temperature-β-glycosidase and a high temperature-α-L-arabinofuranosidase.

2. The composition for production of ginsenoside compound K according to claim 1,
wherein the high temperature-β-glycosidase is a β-glycosidase of Sulfolobus solfataricus, and the high temperature-α-L-arabinofuranosidase is an α-L-arabinofuranosidase of Thermotoga petrophila.

3. The composition for production of ginsenoside compound K according to claim 1,
wherein the content of the high temperature-α-L-arabinofuranosidase is 1 part by weight or more based on 100 parts by weight of the high temperature-β-glycosidase.

4. The composition for production of ginsenoside compound K according to claim 1,
wherein the content of the high temperature-α-L-arabinofuranosidase is 2.5 parts by weight or more based on 100 parts by weight of the high temperature-β-glycosidase.

5. The composition for production of ginsenoside compound K according to claim 2,
wherein the high temperature-β-glycosidase is an enzyme consisting of the amino acid sequence of SEQ ID NO: 2 and the high temperature-α-L-arabinofuranosidase is an enzyme consisting of the amino acid sequence of SEQ ID NO: 4.

6. A method for preparing the composition for production of ginsenoside compound K according to claim 1,
wherein the method comprises expression in E. coli transformed with a vector comprising the base sequence of SEQ ID NO: 3; and a vector comprising the base sequences of SEQ ID NO:
13 and SEQ ID NO: 14.

7. A method for preparing ginsenoside compound K, comprising the step of fermenting a saponin-containing material comprising at least one of ginsenoside Rb 1, ginsenoside Rb2, ginsenoside Rc, and ginsenoside Rd with a high temperature-β-glycosidase and a high temperature-α-L-arabinofuranosidase.

8. The method for preparing ginsenoside compound K according to claim 7,
wherein the step of fermentation is fermentation using the composition for production of ginsenoside compound K according to claim 1.

9. The method for preparing ginsenoside compound K according to claim 7,
wherein the step of fermentation is applying each of a high temperature-β-glycosidase and a high temperature-α-L-arabinofuranosidase.

10. The method for preparing ginsenoside compound K according to claim 9,
wherein the high temperature-β-glycosidase is a β-glycosidase of Sulfolobus solfataricus, and the high temperature-α-L-arabinofuranosidase is an α-L-arabinofuranosidase of Thermotoga petrophila.

11. The method for preparing ginsenoside compound K according to claim 9,
wherein the high temperature-α-L-arabinofuranosidase is applied in an amount of 1 part by weight or more based on 100 parts by weight of the high temperature-β-glycosidase.

12. The method for preparing ginsenoside compound K according to claim 7,
wherein the saponin-containing material is red ginseng extract.

13. The method for preparing ginsenoside compound K according to claim 7,
wherein the fermentation is fermentation at a temperature of 70° C. to 95° C.

14. The method for preparing ginsenoside compound K according to claim 7,
wherein the fermentation is fermentation at a temperature of 80° C. to 90° C.
